(19) United States
(12) Patent Application Publication
(10) Pub. No.: US 2015/0355139 A1
(43) Pub. Date: Dec. 10, 2015

(54) GAAS-BASED DETECTOR HIGHLY STABLE IN AQUEOUS SOLUTIONS

(71) Applicant: YEDA RESEARCH AND DEVELOPMENT CO. LTD, Rehovot (IL)

(72) Inventors: Ron NAAMAN, Yarkona (IL); Maria TKACHEV, Rehovot (IL); Anand Kumar TATIKONDA, Rehovot (IL); Eyal CAPUA, Rehovot (IL)

(21) Appl. No.: 14/762,081

(22) PCT Filed: Jan. 21, 2014
(86) PCT No.: PCT/IL2014/050071

§ 371 Date: Jul. 20, 2015

Related U.S. Application Data
(60) Provisional application No. 61/754,763, filed on Jan. 21, 2013.
(60) Provisional application No. 61/890,988, filed on Oct. 15, 2013.

(51) Int. Cl. G01N 27/414; G01N 33/543; H01L 21/02; H01L 29/20; H01L 21/306

(52) U.S. Cl. CPC G01N 27/4145; H01L 29/20; H01L 21/30612; H01L 21/02118; H01L 21/02282; G01N 27/4148; G01N 33/54373

(57) ABSTRACT

The present invention provides semiconductor devices, particularly devices based on the Molecular Controlled Semiconductor Resistor (MOCSER), which are highly stable in aqueous solutions. The semiconductor devices of the invention may be used for the detection of various target molecules, e.g., proteins, peptides, carbohydrates and small molecules, in different solutions such as physiological solution, bodily fluids and bodily fluid-based solutions.

### TECHNICAL FIELD

The present invention provides semiconductor devices, particularly devices based on molecular controlled semiconductor resistors, highly stable in aqueous solutions.

### BACKGROUND ART

Recent advances in microelectronics, electrochemistry, and nanotechnology make it possible to develop semiconductor-based sensors for a wide variety of applications, among them biosensors (Arregui, 2009; Makowski and Ivanisevic, 2011; Dahlin et al., 2012; Aqua et al., 2008). Biosensors typically combine biological elements with a physicochemical transducer (Thévenot et al., 2001). Semiconductor devices based on transistor-like structures are ideal candidates for biosensing applications due to their low production cost, small size, and direct conversion of the sensing event to changes in electrical current. Specifically, gallium arsenide (GaAs)-based sensors have interesting properties owing to the high mobility of the charge carriers and the high sensitivity to surface potential changes (Gartsman et al., 1998). Moreover, the use of GaAs makes it possible to design heterostructures with special electronic properties, such as 2D electron gas and quantum wells (Delagebeaudeuf et al., 1980). One of the promising platforms for biosensing is the Molecular Controlled Semiconductor Resistor (MOCSER) (International Patent Publication No. WO 98/19151, herewith incorporated by reference in its entirety as if fully disclosed herein; Gartsman et al., 1998; Capua et al., 2009a), based on GaAs Pseudomorphic High Electron Mobility Transistor (pHEMT), which was shown to be applicable for various sensing applications (Vilar et al., 2006; Capua et al., 2009b; Bavli et al., 2012). Due to their electronic properties, GaAs-based sensors were found to be superior to silicon-based devices in terms of sensitivity (Capua et al., 2009a; Naaman, 2011).

In general, producing a stable semiconductor surface for chemical sensing is challenging, since most semiconductors tend to oxidize under ambient conditions, some of them in a non-reproducible manner. In case of GaAs, this issue is of critical importance since GaAs is known to have a chemically unstable surface. The chemical stability issue is especially severe in aqueous environments, where the material is continuously etched due to rapid oxide dissolution in water (Vilar et al., 2005).

The need to develop a reliable method for passivation of GaAs surfaces was realized long ago (Green and Spicer, 1993). It is especially important for any in vivo application, in which one has to protect the GaAs surface and prevent toxic arsenic compounds from penetrating into the living system. Various surface-modification techniques were explored for this purpose (Seker et al., 2000), among them inorganic sulfide treatments (Bessolov et al., 1998; Konenkova, 2002), adsorption of organic thiols and sulfides (Lunt et al., 1991), and deposition of thiol-based self-assembled monolayers (SAM) (Ding and Dubowski, 2005; Arudra et al., 2012). However, none of these methods results in a stable enough surface that allows the device to be operated under physiological conditions for several hours.

Hou et al. (1997) employed chemical cross-linking of 3-mercaptopropyl trimethoxysilane (MPTMS) after depositing MPTMS SAM on GaAs in order to improve the stability of the coating. This idea was further developed by Kirchner et al. (2002) to achieve functionalization of the GaAs surface by polymerization of MPTMS in a solution sol-gel process. The resulting polymer coating protects the GaAs substrate from etching in water (Kirchner et al., 2002).

International Patent Publication No. WO 2012/168932, herewith incorporated by reference in its entirety as if fully disclosed herein, discloses a MOCSER-based detector in which the above method was implemented, more particularly, a device for the detection of an active site-containing protein or a ligand thereof in a solution, said device comprising at least one insulating or semi-insulating layer; at least one conducting semiconductor layer on top of one of said insulating or semi-insulating layers; two conducting pads on both sides on top of the upper layer making electrical contact with said at least one conducting semiconductor layer; a protective molecular layer fabricated on top of said upper layer, protecting said layer from corrosion; and said ligand or active site-containing protein linked to said protective molecular layer, wherein exposure of said ligand or active site-containing protein to a solution containing said active site-containing protein or ligand, respectively, causes a current change through the device when a constant electric potential is applied between the two conducting pads. However, as found, although coating with thin MPTMS film allows corrosion protection when immersed in water, and permits electrical measurements with GaAs-based devices up to a few hours, these devices lack long-term stability. The MPTMS polymer layer is not stable enough for prolonged electrical measurements, leading to device degradation (Kirchner et al., 2002). Apparently, the degradation is caused by water penetrating through tiny pinholes in the protecting polymer layer. Probably these pinholes grow in size during device operation since it is heated by the applied current, which results in etching of the GaAs at the device-polymer interface.

### SUMMARY OF INVENTION

In one aspect, the present invention provides a semiconductor device comprising at least one conducting semiconductor layer, optionally at least one insulating or semi-insulating layer, and a protective organic molecular layer fabricated on top of an upper layer which is either one of said at least one conducting semiconductor layer or one of said at least one insulating or semi-insulating layer, protecting said upper layer from corrosion, said protective organic molecular layer is configured such that when in contact with an aqueous solution, said semiconductor device is sensitive to pH changes in said solution both when fresh and following application of a constant electrical potential of 1 Volt through said at least one conducting semiconductor layer for a period of time of at least 10 hours.

In a particular such aspect, the present invention provides such a device configured as the MOCSER disclosed in WO 98/19151, i.e., a semiconductor device as defined above, comprising at least one conducting semiconductor layer, at least one insulating or semi-insulating layer, two conducting pads, and a protective organic molecular layer, wherein said at least one conducting semiconductor layer is on top of one of said at least one insulating or semi-insulating layer, said two conducting pads are on both sides on top of an upper layer which is either one of said at least one conducting semiconductor layer or one of said at least one insulating or semi-insulating layer, making electrical contact with said at least one conducting semiconductor layer, and said protective organic molecular layer is fabricated on top of said upper layer.

The semiconductor device of the invention may be used for the detection of a target molecule in a solution. In another aspect, the present invention thus relates to a method for the detection of a target molecule in a solution, said method comprising: (i) exposing a semiconductor device as defined above, when configured as a MOCSER, to said solution; and (ii) monitoring the presence of said target molecules in said solution according to the changes in the current measured in said semiconductor device when a constant electric potential is applied between the two conducting pads.

A particular such devices capable of detecting a target molecule in a solution is configured as a MOCSER and further comprises a layer of multifunctional organic molecules capable of binding said target molecule via a functional group thereof, wherein said layer of multifunctional organic molecules is linked either directly or indirectly to said protective organic molecular layer, and exposure of said multifunctional organic molecules to a solution containing said target molecule causes a current change through the semiconductor device when a constant electric potential is applied between the two conducting pads. In a further aspect, the present invention thus relates to a method for the detection of a target molecule in a solution, said method comprising: (i) exposing a semiconductor device as defined above, when configured as a MOCSER and further comprises a layer of multifunctional organic molecules capable of binding said target molecule via a functional group thereof, to said solution; and (ii) monitoring the presence of said target molecules in said solution according to the changes in the current measured in said semiconductor device when a constant electric potential is applied between the two conducting pads.

The semiconductor device of the invention may also be used for the detection of an active site-containing protein or a ligand thereof in a solution. A particular such device is configured as a MOCSER and further comprises said ligand or active site-containing protein, wherein said ligand or active site-containing protein is linked either directly or indirectly to said protective organic molecular layer, and exposure of said ligand or active site-containing protein, to a solution containing said active site-containing protein or ligand, respectively, causes a current change through the semiconductor device when a constant electric potential is applied between the two conducting pads.

In yet another aspect, the present invention thus relates to a method for the detection of an active site-containing protein or a ligand thereof in a solution, said method comprising: (i) exposing a semiconductor device as defined above, when configured as a MOCSER and further comprises said ligand or active site-containing protein, to said solution; and (ii) monitoring the presence of said active site-containing protein or ligand in said solution according to the changes in the current measured in said semiconductor device when a constant electric potential is applied between the two conducting pads.

The methods of the invention may further be used for quantification of the analyte detected, i.e., said target molecule, or active site-containing protein or ligand thereof, in said solution, wherein the current change through the semiconductor device when a constant electric potential is applied between the two conducting pads is proportional to the concentration of said analyte in the solution.

### BRIEF DESCRIPTION OF DRAWINGS

FIGS. 1A-1D show AFM images of polymer layer prepared under the standard conditions with 0.4 vol. % MPTMS in EtOH solution on an n-type GaAs sample (1A, 1B) and GaAs pHEMT-based MOCSER device (1C, 1D). (1A, 1C) Amplitude images of 5×5 μm scans show similar polymer agglomerating sites both on the MOCSER and GaAs substrates. (1B, 1D) 3×3 μm scans of defect-free areas where the roughness analysis was performed.

FIGS. 2A-2B show a schematic representation of the GaAs Pseudomorphic High Electron Mobility Transistor (pHEMT) structure used for the MOCSER device fabrication (2A); and a schematic representation of the experimental setup (2B). A peristaltic pump was used to transfer analyte samples to the MOCSER sensing area inside a PDMS-based flow cell. An Ag/AgCl reference electrode was connected via a salt bridge.

FIG. 3 shows AFM image of non-continuous layer obtained when polymerization occurs at a low concentration of 0.1 vol. % MPTMS in EtOH.

FIGS. 4A-4B show AFM images (3×3 μm) of MOCSER devices coated with MPTMS by the standard procedure (0.4 vol. % solution of MPTMS) before (4A) and after (4B) electrical measurements in aqueous environments for several hours. Surface roughness (peak-to-peak and RMS values) increased after the measurements. The RMS value increased from 1.43 nm to 2.58 nm (note the difference in scale between 4A and 4B).

FIGS. 5A-5B show AFM images of GaAs samples with MPTMS polymer deposited in a solution of 0.4 vol. % MPTMS. (5A) The same concentration was used for the primary layer (8 hours) and for the polymer layer adsorption. (5B) The primary layer deposited in 0.1 vol. % MPTMS solution for 8 hours and the polymer layer adsorbed in 0.4 vol. % MPTMS solution. No significant difference in surface roughness was observed (samples were prepared on the same day).

FIGS. 6A-6B show AFM images of GaAs samples with the MPTMS polymer deposited in a solution of 0.3 vol. % MPTMS. (6A) The same concentration was used for the primary layer (4 hours) and for the polymer layer adsorption (standard procedure). (6B) The primary layer deposited in a solution of 0.1 vol. % MPTMS for 8 hours and the polymer layer adsorbed in 0.3 vol. % MPTMS solution. The roughness significantly decreases with the modified procedure. There are numerous point defects in (6A); here roughness analysis was performed on defect-free areas and not on the whole scan (the samples were prepared on the same day).

FIGS. 7A-7B show the response of MOCSER devices coated with MPTMS by the standard procedure (0.3 vol. % MPTMS for both 4-hour primary layer adsorption and for the polymerization step). (7A) Freshly prepared device exhibits good response (average on 10 channels). (7B) After 7 hours of operation, 4 channels fail, and a lower response is exhibited (average on 6 working channels).

FIGS. 8A-8B show that the MOCSER response to pH changes in case of MPTMS deposited according to the new procedure (0.1 vol. % MPTMS for 8 hours of primary layer adsorption, 0.3 vol. % MPTMS for the polymerization step). (8A) Freshly prepared device exhibits good performance (average on 8 channels). (8B) The same 8 channels still working after 15 hours of operation and exhibit good sensitivity to pH changes.

FIGS. 9A-9B show the response to pH of MOCSER devices coated with MPTMS by the new procedure with NH₄OH dispersed in MPTMS polymerization solution by Vortex (0.1 vol. % MPTMS for 8 hours of primary layer adsorption, 0.3 vol. % MPTMS for the polymerization step). (9A) Freshly prepared device (average on 10 channels). (9B) After 7 hours of operation (after on 9 channels).

FIGS. 10A-10D show AFM images (amplitude images of 13×13 μm scan) of the polymer layer prepared under the standard conditions with a 0.3 vol. % MPTMS solution (10A, 10B) and with new procedures—0.1 vol. % MPTMS solution for the primary layer deposition for 8 hours, and 0.3 vol. % MPTMS solution for the polymerization step (10C, 10D). (10A) Freshly deposited MPTMS according to the standard procedure on an n-type GaAs sample; (10B) MOCSER device surface coated according to the standard procedure after electrical measurements of the device for 15 hours. The density of the polymer irregularities increased after the device was operated under electrical stress in an aqueous environment. (10C) Freshly deposited MPTMS according to the new procedure on an n-type GaAs sample. A very low density of surface irregularities is observed. (10D) The MOCSER device surface coated according to the new procedure after taking electrical measurements of the device. The number of polymer irregularities increased after the device was operated for 15 hours, but the overall density of these defects is much lower than in 10B and is comparable to that of the device freshly prepared by the standard procedure.

FIG. 11 shows a schematic representation of the experimental setup used in Study 2. A syringe pump was used to transfer analyte samples to a GaAs-based MOCSER on top of which a PDMS-based microfluidic flow cell was constructed. An Ag/AgCl reference (Ref.) electrode was connected via a salt bridge. Human hemoglobin (Hb) antibodies (Hb Ab) were attached to the MPTMS-APTMS modified GaAs surface through Protein G, followed by BSA blocking of the non-binding sites.

FIG. 12 shows the normalized change in the MOCSER source-drain current as a function of time upon exposure to different concentrations of hemoglobin, indicated (as mg/ml) in italic, dissolved in phosphate buffer (50 mM). The sample flow rate is 0.02 ml/min. [symbol] and [symbol] indicate the time of exposure to the analyte and to phosphate buffer (50 mM, washing), respectively. The gradient of the normalized response (nA/sec) is shown in bold. I and Iₒ are the measured current and the baseline current, respectively.

FIG. 13 shows the normalized change in the MOCSER source-drain current as a function of time upon exposure to different concentrations of hemoglobin, indicated (as mg/ml) in italic, dissolved in urine. The sample flow rate is 0.02 ml/min. [symbol] and [symbol] indicate the time of exposure to the analyte and to phosphate buffer (50 mM, washing), respectively. The gradient of the normalized response (nA/sec) is shown in bold. I and Iₒ are the measured current and baseline current, respectively.

FIG. 14 shows the normalized response of the MOCSER device to different concentrations of hemoglobin (Hb) in phosphate buffer (50 mM) and in urine. The response is defined as the gradient of the change in the MOCSER source-drain current during the first 150 seconds after injection of the analyte, as indicated in FIGS. 12 and 13. Four independent studies were performed and the error is based on the variation in the results in those studies.

FIG. 15 shows the normalized change in the MOCSER source-drain current as a function of time upon exposure to hemoglobin, indicated (as mg/ml) in italic, dissolved in a fluid obtained from ERCP measurements and diluted 25-fold. The sample flow rate is 0.02 ml/min. [symbol] and [symbol] indicate the time of exposure to the analyte and to ERCP diluted 25-fold (washing), respectively. The gradient of the normalized response is shown in bold. I and Iₒ are the measured current and the baseline current, respectively.

FIG. 16 shows the response of an array-based sensor composed of three devices, wherein the first one is coated with APTMS with NH₂ groups exposed to the analytes; the second one is modified with Protein G and BSA with no hemoglobin antibodies (No Ab); and in the third one, hemoglobin antibodies are bound to the Protein G and the rest of the surface is blocked with BSA (With Ab). The array was exposed to hemoglobin (0.25 mg/ml) dissolved in either buffer solution (Hb in buffer) or urine (Hb in urine), and to avidin (0.25 mg/ml) dissolved in either buffer or urine. The response of the device is defined as [(I−Iₒ)×10²/Iₒ] and each rectangle represents the response as a function of time, wherein the full width of the rectangles corresponds to 150 sec.

FIG. 17 shows a normalized change in the MOCSER source-drain current as a function of time upon sequential exposure to HEPES buffer (50 mM), 0.2 mg/ml of Protein G, 0.1 mg/ml of BSA and 0.1 mg/ml of hemoglobin antibodies, with a 0.02 ml/min flow rate. [symbol] and [symbol] indicate the time of exposure to the respective analyte solution and to phosphate buffer (50 mM, washing), respectively.

FIGS. 18A-18C show (18A) a schematic representation of the experimental setup used in Study 3. A syringe pump was used to transfer analyte samples to a GaAs-based MOCSER on top of which a PDMS-based microfluidic flow cell was constructed. An Ag/AgCl reference electrode was connected via a salt bridge. The MPTMS polymerized on the surface of the MOCSER is terminated with SH groups that are partially negatively charged and interact with the analyte. A dialysis membrane was placed on top of the sensing area allowing the diffusion of only small molecules to the surface; (18B) a picture of the circuitry board in a dual Faraday box; and (18C) a wire-bonded MOCSER chip encapsulated with PDMS and connected to capillary tubes.

FIG. 19 shows human HeLa cells cultured for 24 hours on petri dish, bare GaAs, GaAs coated with MPTMS, or GaAs coated with MPTMS\APTMS, demonstrating that GaAs enables the growth of the cells.

FIGS. 20A-20B show the normalized response of the MOCSER device to different concentrations of ammonia at pH 1.2 (20A) and pH 5 (20B). The response is defined as the gradient of the change in the MOCSER source-drain current as a function of time. Normalized response was calculated by the change in the current, i.e., the slope, for 150 sec from the time of injection of the analyte. [symbol] indicates the time of exposure to the analyte, and [symbol] indicates washing of the analyte.

FIGS. 21A-21B shows the normalized change in the MOCSER source-drain current as a function of time upon exposure to 10 ppm of ammonia dissolved in water at pH 3, wherein the device surface is coated with MPTMS polymer where SH groups are exposed to the analytes (21A); or further coated with APTMS where NH₃ groups are exposed to the analytes (21B). As shown, the response to ammonia, when the MOCSER is coated with APTMS, is opposite in sign to that observed when the surface is coated with MPTMS. [symbol] , [symbol] and [symbol] indicate ammonia in water, ammonia at pH 3, and washing, respectively.

FIGS. 22A-22B show the normalized change in the MPTMS-coated MOCSER source-drain current to different concentration of ammonia in water. The normalized current measured upon exposure of the device to different concentrations of ammonia (22A). The change in the normalized current measured upon exposure to ammonia plotted as a function of ammonia concentration (logarithmic scale) (22B). [symbol] and [symbol] indicate the time of exposure to the analyte and to water (washing), respectively.

FIGS. 23A-23C show the change in the MOCSER source-drain current as a function of the solution pH when the device is coated with APTMS and exposed to 10 or 2 ppm ammonia (23A, 23B, respectively), and when the device is coated with MPTMS and exposed to 10 ppm ammonia (23C).

FIGS. 24A-24B show (24A) the normalized response in the current through the APTMS-coated MOCSER to different concentrations of ammonia at pH 4. The inserts show that at low concentrations (insert a) the change in the current is positive and linearly dependent on the ammonia concentration as there is no substantial pH change of the analyte with addition of ammonia; and at higher concentrations (insert b) the change in current is negative as the pH of the analyte is higher than the pI of APTMS surface. 24B shows the normalized response of the MPTMS-coated MOCSER to different concentrations of ammonia at pH 4. The change in the current is negative and the response is linear until 50 ppm and then the signal saturates.

FIG. 25 shows the response of the MPTMS-coated MOCSER to different concentrations of ammonia in different gastric simulation fluids conditions, i.e., different pH and different salt concentrations. The response of the MOCSER depends more on the pH of the analyte than on the salt concentration. All the responses are negative.

FIGS. 26A-26B show the normalized change in the MOCSER source-drain current as a function of time when exposed to ammonia dissolved in unfiltered (26A) or filtered (26B) gastric fluids obtained from patients either positive or negative for H. pylory. [symbol] and [symbol] indicate the time of exposure to the analyte and to washing, respectively.

FIG. 27 shows the normalized change in the MPTMS-coated MOCSER source-drain current as a function of time when exposed to gastric fluid positive for H. pylory, wherein the signal from gastric fluid negative for H. pylory serves as a baseline. A dialysis membrane was used to avoid blocking of the sensing area by the agglomerated macromolecules. The pH of the gastric fluid from the patience is between pH 1 to 2. [symbol] and [symbol] indicate the time of exposure to gastric fluid positive or negative to H. pylory, respectively. The response after washing does not decay back to zero since with when the membrane is in place, the diffusion from the surface to the solution is very slow. The variation in the signal for negative samples is less than 10% from the response measured for samples that are positive.

FIGS. 28A-28B show the normalized change in the MPTMS-coated MOCSER source-drain current as a function of time (28A) upon exposure to different concentrations of ammonia dissolved in gastric fluids obtained from patients (pH 1-2), and as a function of ammonia concentration (28B). A dialysis membrane was placed on the MOCSER sensing area to avoid the agglomerated macromolecules in the gastric fluid to block the sensing area. [symbol] and [symbol] indicate the time of exposure to the analyte solution and to gastric simulation (washing), respectively.

FIGS. 29A-29B show the normalized change in the MOCSER source-drain current as a function of time upon exposure to ammonia dissolved in a gastric fluid, wherein the gastric fluid is filtered and diluted (29A); or is unfiltered and a dialysis membrane is deposited on the surface of the sensing area (29B). [symbol] and [symbol] indicate the time of exposure to the analyte solution (obtained from a H. pylori positive patient), and to a solution known to be negative for H. pylory, respectively.

### DETAILED DESCRIPTION OF THE INVENTION

According to the procedure developed by Hou et al. (1997) and improved by Kirchner et al. (2002) for depositing a protective layer of MPTMS on GaAs substrates, herein also referred to as “the standard procedure”, GaAs substrates are first cleaned in isopropanol, acetone and ethanol for 10 minutes each; oxidized by UV/ozone (UVOCS) for 10 minutes; and are then etched for 5 seconds in hydrofluoric acid (HF 2%), rinsed in deionized water (DDW), etched for 30 seconds in ammonium hydroxide (NH₄OH 25%), and finally rinsed in DDW again. After etching, the substrates are dried with nitrogen and immediately immersed in ethanol solution of MPTMS, wherein in previous works, concentration of either 0.3 vol. % (16 mM) (Bavli et al., 2012) or 0.4 vol. % (21.5 mM) (Tatikonda et al., 2013) were used for protecting GaAs-based MOCSER devices. Placing in a water bath at 50° C. for 4 hours allows primary MPTMS layer adsorption with stable thiol binding to the substrate and with the reactive methoxy groups pointing outwards. Next, polymerization of MPTMS is initiated by adding NH₄OH (25%), acting as a condensation agent, in an amount adjusted to the concentration of MPTMS in the adsorption solution, i.e., 3 vol. % NH₄OH for a 0.3 vol. % MPTMS concentration, and 4 vol. % NH₄OH for 0.4 vol. % MPTMS solution. The solution is kept at 50° C. for an additional 16 hours, and the samples are then rinsed with ethanol and dried under a stream of nitrogen.

It has now been found, in accordance with the present invention, that by separating the two steps of the standard procedure, i.e., the adsorption of a primary MPTMS layer on the substrate and the MPTMS polymerization, and varying the conditions for the primary layer adsorption, a smooth primary MPTMS layer with significantly better adhesion to the GaAs substrate is formed, and consequently a more uniform and thinner polymer layer is deposited during the second step. This modified process is also herein referred to as “the new procedure”.

As shown in the Experimental section hereinafter, when the polymer deposition is performed in a solution of 0.4 vol. % MPTMS, the thickness of the layer obtained ranges from 20 to 30 nm, whereas in case the process is performed in a solution of 0.3 vol. % MPTMS, the thickness of the resulting layer decreases to 15-25 nm. In addition, while changing the primary MPTMS layer adsorption conditions does not affect the quality of the polymer layer obtained in a solution of 0.4 vol. % MPTMS, for samples prepared in a 0.3 vol. % MPTMS solution, the primary MPTMS adsorbed layer profoundly affects the overall polymer quality. Atomic force microscope (AFM) images clearly demonstrate that when the primary MPTMS layer is adsorbed from solutions with low concentrations and long deposition times, the peak-to-peak and root mean square (RMS) values are significantly reduced, indicating the role of the primary layer in improving the surface quality.

As shown in Study 1 hereinafter, in order to compare the stability and sensitivity of semiconductor devices protected according to the new procedure with those of semiconductor devices protected according to the standard procedure, MOCSER devices coated with MPTMS under different conditions were prepared and the changes in the signal as a function of time upon exposure to phosphate buffer solutions having different pH values were measured, first on freshly prepared devices and then after 7-15 hours of continuous operation during which a constant electrical potential of 1 Volt was applied through the device.

As generally found, devices coated with a polymer prepared in 0.4 vol. % MPTMS solutions were remarkably less stable than devices prepared in 0.3 vol. % MPTMS solutions, wherein some of the former stopped working already during the initial pH measurements, and those that survived after 12 hours lost their sensitivity. Devices prepared according to the new procedure were sensitive to pH changes both when fresh and after 12 hours of measurements, though the sensitivity was lower than that observed for the 0.3 vol. % MPTMS polymerization and the overall device's performance decreased with time (high noise). A MOCSER device coated with a polymer prepared in 0.3 vol. % MPTMS solution according to the standard procedure exhibited high sensitivity when fresh, but only 6 out of 10 operating channels survived the 7-hour measurements and the device sensitivity decreased. After 15 hours of continuous measurements, most of the devices failed.

In sharp contrast, in devices coated with MPTMS according to the new procedure, all 8 channels that worked at the beginning of the experiment exhibited stable performance and were still sensitive to pH changes after 15 hours of continuous measurements. A similar device coated with a polymer prepared by the new procedure when slightly modified, more particularly wherein the polymerization step was performed in 0.3 vol. % MPTMS solution with NH₄OH dispersed by Vortex, exhibited a significantly higher sensitivity due to the thinner polymer layer obtained, when fresh, wherein 9 out of 10 operating channels were still sensitive after 7 hours of measurements, but only 6 channels functioned properly, although with reduced sensitivity, after 15 hours of measurements.

In order to analyze the effect of the continuous electrical measurements on the structure of the protecting polymer prepared in 0.3 vol. % MPTMS solution, AFM images of the protecting layer were used. As shown in those images, in the case of MOCSER devices coated with a polymer prepared according to the standard procedure, the density of the surface defects significantly increased after the electrical measurements, and roughness analysis revealed that both the peak-to-peak and RMS values dramatically increased as well. In sharp contrast, in devices coated with a polymer prepared according to the new procedure, i.e., adsorption of primary MPTMS layer in 0.1 vol. % MPTMS solution for 8 hours, and 0.3 vol. % MPTMS solution for polymerization, a significantly lower number of surface defects was observed on the surface of the fresh samples, and although increased after the continuous operation, the number of surface defects was still remarkably lower than that observed for devices coated by the standard procedure.

The results of Study 1 indicate why the MPTMS layer deposited on the GaAs devices according to the standard procedure fail to protect. Apparently, pinholes are formed in the layer due to the increased temperature of the operated device, and solutions penetrating through these pinholes etch the GaAs surface of the devices and eventually cause them to malfunction. In contrast, the new procedure provides a more uniform coating with better adhesion to the GaAs substrate, and this coating is significantly less sensitive to the temperature effect, and therefore allows continuous electrical measurements in physiological solutions for more than 15 hours and enables the operation of the sensor in aqueous environments even at very low pH for periods exceeding 24 hours. Additional modifications of the new procedure with better dispersion of NH₄OH result in reduced thickness and consequently significantly higher sensitivity of the protecting layer, but reduced stability as well.

In one aspect, the present invention thus provides a semiconductor device comprising at least one conducting semiconductor layer, optionally at least one insulating or semi-insulating layer, and a protective organic molecular layer fabricated on top of an upper layer which is either one of said at least one conducting semiconductor layer or one of said at least one insulating or semi-insulating layer, protecting said upper layer from corrosion, said protective organic molecular layer is configured such that when in contact with an aqueous solution, said semiconductor device is sensitive to pH changes in said solution both when fresh and following application of a constant electrical potential of 1 Volt through said at least one conducting semiconductor layer for a period of time of at least 10 hours.

The phrase “sensitive to pH changes”, as used herein with respect to the semiconductor device of the present invention, means that when in contact with an aqueous solution, and upon application of a constant electrical potential, e.g., of 1 Volt, through said at least one conducting semiconductor layer, said device is sensitive to changes in the pH of said solution, i.e., in the H⁺ concentration in said solution, no matter whether said semiconductor device is a recently manufactured device that has not been used, i.e., fresh, or an already used device following application of a constant electrical potential through said at least one conducting semiconductor device for a period of time of at least 10 hours, preferably at least 12 hours, more preferably at least 15 hours. The device is sensitive to pH changes because the charge on particular functional groups of the protective organic molecular layer facing the solution, e.g., mercapto and hydroxyl groups, varies with pH, and this variation causes a change in the signal measured as a function of time upon exposure of said protective organic molecular layer to solutions with different pH, e.g., in the range of pH 6 to pH 9.

The results of Study 1 indicate that the sensitivity of the semiconductor device of the present invention to pH changes in the solution following application of a constant electrical potential for a period of time as indicated above results from both the thickness of said protective organic molecular layer, which is in a range of 15-25 nm, and the high quality of its surface, i.e., low peak-to-peak and RMS values; but much more important, from the fact that in sharp contrast to devices having a protective organic molecular layer prepared according to the standard procedure, the quality of the surface of said molecular layer does not significantly decreased, i.e., the peak-to-peak and RMS values do not remarkably increased, following application of a constant electrical potential through said at least one conducting semiconductor layer as indicated above.

The semiconductor device of the present invention may thus alternatively be defined as a semiconductor device comprising at least one conducting semiconductor layer, optionally at least one insulating or semi-insulating layer, and a protective organic molecular layer fabricated on top of an upper layer which is either one of said at least one conducting semiconductor layer or one of said at least one insulating or semi-insulating layer, protecting said upper layer from corrosion, wherein said protective organic molecular layer has a thickness in a range of 5-25 nm and a surface having an RMS roughness of up to 3.5 nm following application of a constant electrical potential of 1 Volt through said at least one conducting semiconductor layer for a period of time of at least 10 hours.

In a particular such aspect, the present invention provides a semiconductor as defined above, configured as the MOCSER, i.e., a semiconductor device as defined above, comprising at least one conducting semiconductor layer, at least one insulating or semi-insulating layer, two conducting pads, and a protective organic molecular layer, wherein said at least one conducting semiconductor layer is on top of one of said at least one insulating or semi-insulating layer, said two conducting pads are on both sides on top of an upper layer which is either one of said at least one conducting semiconductor layer or one of said at least one insulating or semi-insulating layer, making electrical contact with said at least one conducting semiconductor layer, and said protective organic molecular layer is fabricated on top of said upper layer.

In one embodiment, the semiconductor device of the present invention is configured as the MOCSER and composed of at least one insulating or semi-insulating layer, one conducting semiconductor layer, two conducting pads, and a protective organic molecular layer, wherein said conducting semiconductor layer is on top of one of said insulating or semi-insulating layers, said two conducting pads are on both sides on top of an upper layer which is either said conducting semiconductor layer or one of said insulating or semi-insulating layers, making electrical contact with said conducting semiconductor layer, and said protective organic molecular layer is fabricated on top of said upper layer.

The various conducting semiconductor and insulating or semi-insulating layers of the semiconductor device of the present invention are defined as in the basic MOCSER disclosed in WO 98/19151.

In certain embodiments, each one of the conducting semiconductor layers in the semiconductor device of the present invention independently is a semiconductor selected from a III-V and a II-VI material, or mixtures thereof, wherein III, V, II and VI denote the Periodic Table elements III=Al, Ga, In; V=As, P; II=Cd, Zn; VI=S, Se, Te. In particular such embodiments, each one of the conducting semiconductor layers is doped GaAs, doped (Al,Ga)As, or doped (In,Ga)As.

In certain embodiments, each one of the insulating or semi-insulating layers in the semiconductor device of the present invention independently is a dielectric material selected from silicon oxide, silicon nitride or an undoped semiconductor selected from a III-V and a II-VI material, or mixtures thereof, wherein III, V, II and VI denote the Periodic Table elements III=Al, Ga, In; V=As, P; II=Cd, Zn; VI=S, Se, Te. In particular such embodiments, the undoped semiconductor is undoped GaAs, undoped (Al,Ga)As, or undoped (In,Ga)As.

In one particular embodiment exemplified herein, the semiconductor device of the present invention is composed of a first insulating or semi-insulating layer of undoped GaAlAs which is on top of a first conducting semiconductor layer of doped GaAs, said first conducting semiconductor layer is on top of a second insulating or semi-insulating layer of undoped GaAlAs which is on top of a third insulating or semi-insulating layer of undoped InGaAs, said third insulating layer is on top of a fourth insulating or semi-insulating layer of GaAs, wherein on top of said first insulating or semi-insulating layer is a second conducting semiconductor layer of GaAs on top of which is an upper insulating or semi-insulating layer of GaAs, and said protective organic molecular layer is fabricated on top of said upper insulating or semi-insulating layer.

In certain embodiments, the protective organic molecular layer of the semiconductor device of the present invention, in any one of the configurations defined above, comprises a primary layer and a secondary polymer layer. In order to guarantee that the protective organic molecular layer would not interfere with, i.e., reduce, the sensitivity of the device, both the primary layer and the secondary polymer layer are formed by molecules having low dielectric constant so as to eliminate electrical screening of the semiconductor surface from charge on the surface of the organic film.

In particular such embodiments, the primary layer is formed by deposition of an alkoxysilane of the general formula HS—R₁—Si(OR₂)₃ or H₂N—R₁—Si(OR₂)₃, or a mixture thereof, wherein R₁ each independently is a (C₁-C₇)alkylene, preferably (C₃-C₄)alkylene, more preferably propylene, optionally interrupted with one or more —NH— groups, and R₂ each independently is (C₁-C₄)alkyl, preferably methyl or ethyl; and the secondary polymer layer is obtained upon polymerization under basic conditions of an alkoxysilane as defined above or of the general formula R₁′—Si(OR₂)₃ or HSi(OR₂)₃, a tetraalkyl orthosilicate of the general formula Si(OR₂)₄, a biotinylated from thereof, or a mixture of the aforesaid, wherein R₁′ is a (C₁-C₇)alkyl, preferably (C₃-C₄)alkyl, more preferably propylene, optionally interrupted with one or more —NH— groups; and R₂ is as defined above. In these cases, the semiconductor device of the invention thus comprises a protective organic molecular layer comprising a primary layer and a secondary polymer layer, wherein said primary layer comprises a silane moiety of the formula —S—R₁—Si(OH)₃, —S—R₁—Si(OH)₂O—, —S—R₁—Si(OH)(O—)₂, —S—R₁—Si(O—)₃, —NH—R₁—Si(OH)₃, —NH—R₁—Si(OH)₂O—, —NH—R₁—Si(OH)(O—)₂, —NH—R₁—Si(O—)₃, or a mixture thereof; said secondary polymer layer is obtained upon polymerization under basic conditions of an alkoxysilane of the formula HS—R₁—Si(OR₂)₃, H₂N—R₁—Si(OR₂)₃, R₁′—Si(OR₂)₃ or HSi(OR₂)₃, a tetraalkyl orthosilicate of the formula Si(OR₂)₄, a biotinylated from thereof, or a mixture of the aforesaid; R₁ each independently is a (C₁-C₇)alkylene, preferably (C₃-C₄)alkylene, more preferably propylene, optionally interrupted with one or more —NH— groups; R₁′ is a (C₁-C₇)alkyl, preferably (C₃-C₄)alkyl, more preferably propylene, optionally interrupted with one or more —NH— groups; R₂ each independently is a (C₁-C₄)alkyl, preferably methyl or ethyl; and said secondary polymer layer is covalently linked to the said primary layer via —Si—O— bonds.

The term “alkyl”, as used herein, typically means a straight or branched hydrocarbon radical, wherein “(C₁-C₄)alkyl” particularly refers to such radicals having 1-4 carbon atoms. Non-limiting examples of such alkyls include methyl, ethyl, n-propyl, isopropyl, n-butyl, sec-butyl, isobutyl, tert-butyl, and the like. The term “(C₁-C₇)alkylene” refers to a straight or branched divalent hydrocarbon radical having 1-7 carbon atoms and include, e.g., methylene, ethylene, propylene, butylene, 2-methylpropylene, pentylene, 2-methylbutylene, hexylene, 2-methylpentylene, 3-methylpentylene, 2,3-dimethylbutylene, heptylene, and the like.

In certain particular such embodiments, said primary layer comprises (i) a silane moiety of the formula —S—R₁—Si(OH)₃, —S—R₁—Si(OH)₂O—, —S—R₁—Si(OH)(O—)₂, —S—R₁—Si(O—)₃, or a mixture thereof, wherein R₁ is —(CH₂)₃—; (ii) a silane moiety of the formula —NH—R₁—Si(OH)₃, —NH—R₁—Si(OH)₂O—, —NH—R₁—Si(OH)(O—)₂, —NH—R₁—Si(O—)₃, or a mixture thereof, wherein R₁ is —(CH₂)₃— or —(CH₂)₄—; or (iii) a silane moiety of the formula —NH—R₁—Si(OH)₃, —NH—R₁—Si(OH)₂O—, —NH—R₁—Si(OH)(O—)₂, —NH—R₁—Si(O—)₃, or a mixture thereof, wherein R₁ is —(CH₂)₂—NH—(CH₂)₃—. Preferred such embodiments are those wherein the primary layer is formed by deposition of MPTMS, i.e., wherein the primary layer comprises a silane moiety of the formula —S—R₁—Si(OH)₃, —S—R₁—Si(OH)₂O—, —S—R₁—Si(OH)(O—)₂, —S—R₁—Si(O—)₃, or a mixture thereof, wherein R₁ is —(CH₂)₃—.

In certain particular such embodiments, said secondary polymer layer is obtained upon polymerization under basic conditions of MPTMS, 3-mercaptopropyl triethoxysilane, N¹-(3-(trimethoxysilyl)propyl)ethane-1,2-diamine, N¹-(3-(triethoxysilyl)propyl)ethane-1,2-diamine, 3-aminopropyltrimethoxysilane (APTMS), 3-aminopropyltriethoxysilane, 4-aminobutyltriethoxysilane, 4-aminobutyl trimethoxysilane, trimethoxypropylsilane, trimethoxyethylsilane, tetramethyl orthosilicate, a biotinylated from thereof, or a mixture of the aforesaid. Preferred such embodiments are those wherein the secondary layer is obtained upon polymerization under basic conditions of MPTMS of APTMS.

According to the present invention, the protective organic molecular layer may be fabricated on top of the upper layer of the semiconductor device by any suitable process, utilizing any technology known in the art, in which a primary layer with good adhesion to the substrate is first deposited on the upper layer of said device, and a secondary uniform and thin polymer layer is then fabricated on top of said primary layer.

In a particular process developed by the inventors of the present invention and exemplified herein, GaAs substrates are cleaned, oxidized and etched as in the standard procedure; and are then dried with nitrogen, and immersed in a solution of 0.1 vol. % MPTMS in ethanol, at 50° C. for 8 hours, for primary layer deposition. For the polymerization step, the MPTMS concentration is increased either to 0.3 or 0.4 vol. % by adding MPTMS to the first step solution, and NH₄OH acting as a condensation agent is then added to initiate the polymerization. Alternatively, a new solution of 0.3 or 0.4 vol. % MPTMS is prepared for the polymerization step, NH₄OH is added, and the mixture is thoroughly shaken with Vortex for 30 seconds for better dispersion of the condensation agent before immersing the sample. In both cases, the polymerization step is carried out at 50° C. for an additional 16 hours, and the GaAs samples are then rinsed with ethanol and dried.

In specific embodiments such as exemplified herein, the semiconductor device of the invention, in any one of the configurations defined above, thus comprises a protective organic molecular layer comprising a primary layer and a secondary polymer layer, wherein said primary layer comprises a silane moiety of the formula —S—R₁—Si(OH)₃, —S—R₁—Si(OH)₂O—, —S—R₁—Si(OH)(O—)₂, —S—R₁—Si(O—)₃, or a mixture thereof, wherein R₁ is —(CH₂)₃—; and said secondary polymer layer is obtained upon polymerization under basic conditions of MPTMS.

In more specific such embodiments, the semiconductor device of the invention comprises a protective organic molecular layer as defined above, formed by a process comprising the steps of:
(i) etching the upper surface of said upper layer;
(ii) immersing the etched surface of said upper layer in a solution of 0.1 vol. % MPTMS in ethanol, at a temperature of about 50° C. for about 8 hours, thereby forming a primary layer of MPTMS moieties deposited on top of said surface of said upper layer; and either:
   a) immersing the surface of said upper layer on which a primary layer of MPTMS moieties is deposited in a solution of 0.3-0.4 vol. % MPTMS in ethanol, followed by the addition of a base such as NH₄OH to thereby initiate polymerization of said MPTMS and MPTMS moieties, at a temperature of about 50° C. for about 16 hours, thereby forming a secondary layer of polymerized MPTMS linked to the said primary layer via —Si—O— bonds; or
   b) immersing the surface of said upper layer on which a primary layer of MPTMS moieties is deposited in a solution of 0.3-0.4 vol. % MPTMS and a base such as NH₄OH in ethanol, wherein said base initiates polymerization of said MPTMS and MPTMS moieties, at a temperature of about 50° C. for about 16 hours, thereby obtaining a secondary layer of polymerized MPTMS linked to the said primary layer via —Si—O— bonds.

In particular such embodiments, the semiconductor device of the invention comprises a protective organic molecular layer formed by the process described above, wherein said protective organic molecular layer has (i) a thickness of 22.3±6.7 nm and a surface having an RMS roughness of 1.5±0.1 nm when fresh and RMS roughness of 2.0±1.4 following application of a constant electrical potential of 1 Volt through said at least one conducting semiconductor layer for a period of time of at least 10 hours; or (ii) a thickness of about 15.9±1.2 nm and a surface having an RMS roughness of 1.9±0.01 nm when fresh and RMS roughness of 1.9±0.1 nm following application of a constant electrical potential of 1 Volt through said at least one conducting semiconductor layer for a period of time of at least 10 hours.

Study 2 hereinafter shows that a MOCSER device as defined above, i.e., protected according to the procedure exemplified in the Experimental section, is highly sensitive for continuous monitoring of hemoglobin; and that by utilizing an array configuration, both high sensitivity and high selectivity can be obtained. The ability to apply semiconductor devices for sensing biological molecules in biological environments opens up the possibility of taking advantages of the microelectronics-based technologies in real-time applications for sensing in vivo. The concentration of hemoglobin in the blood is 130 mg/ml, while for people with hematuria the concentration of hemoglobin in the urine is of about 1 mg/ml (Packham et al., 2005). Study 2 demonstrates that hemoglobin can be sensed in urine with sensitivity of 0.1 mg/ml and the sensitivity is enough even in harsh physiological fluids like bile juice. Hence the selectivity and sensitivity demonstrated make the technique relevant for medical applications. The method described herein combines the sensitivity and selectivity with short measuring time and low production costs, and it is therefore an attractive venue for continuous sensing of various target molecules, e.g., proteins, peptides, carbohydrates and small molecules, in different solutions such as physiological solution, bodily fluids and bodily fluid-based solutions.

Detection of ammonia in gases and liquids is of great importance not just in industrial and environmental safety but also in the human body, as ammonia is one of the main metabolites. There are many different techniques and types of sensors for detecting ammonia (Meyerson et al., 1978; Fraticelli and Meyerhoff, 1981; Bekyarova et al., 2004; Alegret et al., 1990; Meyerhoff and Robins, 1980); however the microelectronic-based sensors are expected to have high potential in applications as they are being based on the established microelectronic technology that allows their inexpensive production as well as their miniaturization and ability to use in large arrays (Jaffrezic-Renault et al., 1999; Senillou et al., 1999; Humenyuk et al., 2006; Liu et al., 2004; Bergveld, 1986). However, semiconductor-based devices, particularly devices based on GaAs, are rapidly oxidized in aqueous solutions and both chemically and electrically unstable; and release bio-incompatible substrates. As a result, there are difficulties in adapting them as bio-sensors and especially in vivo.

There are number of diseases associated with ammonia such as Reye's syndrome (Autret-Lecaa et al., 2001), hyperammonia, stomach cancer and ulcers. Stomach cancer is the most significant and is caused by Helicobacter pylori (H. pylori) infection. H. pylori bacterium reside in the lining of the stomach and release large quantities of ammonia (Chittajallu et al., 1991; Megraud et al., 1992). Present clinical analysis uses biopsy for determining ammonia concentration but the procedure is quite tedious and time consuming. The standard technique currently used to measure the presence of H. pylori is based on a breathing test where urea originated carbon isotopes are being measured. Various types of sensors were proposed for a direct ammonia sensing; however, they suffer from several drawbacks. Sensors based on potentiometric or impedance, e.g., lose their sensitivity because of the applied over potential (Radomska et al., 2004; Mohabbati-Kalejahi et al., 2012; Salimi et al., 2005; http://www.instrumart.com/assets/ISEammonia_manual.pdf). The use of enzymatic-ISFETs was also considered but the enzymes are known to interfere with the sensing area and the drift in the response decreases their sensitivity (Senillou et al., 1999).

Study 3 shows that a MOCSER device as defined above, i.e., protected according to the procedure exemplified in the Experimental section, is capable of sensing ammonia in ex-vivo gastrointestinal fluids. The ability to work in this environment was achieved by protecting the semiconductor device with a thin MPTMS polymer layer which exposes thiol groups, acting as the sensing molecules, towards the analyte. The change in the protonation of the thiols upon exposure to ammonia is transformed directly to a change in the current through the solid-state device. Hence, the sensing and the device protection are performed by the same layer. The interaction of the sensing molecules with the ammonia is electrostatic and does not involve bond formation. Hence, the device can be used continuously with samples containing different concentrations of ammonia. As shown in this study, beyond the protection layer, it is important to protect the sensor from non-specific interactions with species that exist in the raw sample such as proteins and fats. This was achieved by coating the device with an additional membrane that eliminates the access of such proteins to the device's surface.

In certain embodiments, the protective organic molecular layer of the semiconductor device of the present invention, in any of the configurations defined above, comprises a primary layer and a secondary polymer layer, and further comprises a tertiary layer deposited on top of said secondary polymer layer, wherein said tertiary layer comprises an alkoxysilane of the formula HS—R₁—Si(OR₂)₃, H₂N—R₁—Si(OR₂)₃, R₁′—Si(OR₂)₃ or HSi(OR₂)₃, a tetraalkyl orthosilicate of the formula Si(OR₂)₄, or a mixture of the aforesaid; R₁ each independently is a (C₁-C₇)alkylene, preferably (C₃-C₄)alkylene, more preferably propylene, optionally interrupted with one or more —NH— groups; R₁′ is a (C₁-C₇)alkyl, preferably (C₃-C₄)alkyl, more preferably propylene, optionally interrupted with one or more —NH— groups; R₂ each independently is a (C₁-C₄)alkyl, preferably methyl or ethyl; and said tertiary layer is covalently linked to the said secondary polymer layer, e.g., via —Si—O— bonds, —S—S— bond, or both. In particular such embodiments, said alkoxysilane is MPTMS, 3-mercaptopropyltriethoxysilane, N¹-(3-(trimethoxysilyl)propyl)ethane-1,2-diamine, N¹-(3-(triethoxysilyl)propyl)ethane-1,2-diamine, APTMS, 3-aminopropyl triethoxysilane, 4-aminobutyltriethoxysilane, 4-aminobutyltrimethoxysilane, trimethoxy propylsilane, or trimethoxyethylsilane; and said tetraalkyl orthosilicate is tetramethyl orthosilicate. In a more particular such embodiment such as that exemplified in Study 2, said primary layer comprises a silane moiety of the formula —S—R₁—Si(OH)₃, —S—R₁—Si(OH)₂O—, —S—R₁—Si(OH)(O—)₂, —S—R₁—Si(O—)₃, or a mixture thereof, wherein R₁ is —(CH₂)₃—; said secondary polymer layer is obtained upon polymerization under basic conditions of MPTMS; and an tertiary layer of APTMS is deposited on top of said secondary polymer layer.

According to the present invention, the tertiary layer can be deposited on top of the secondary polymer layer using any process or technique known in the art, e.g., by chemical vapor deposition (CVD).

The semiconductor device of the present invention, in any of the configurations defined above, may be used for the detection of a target molecule in a solution, e.g., using—as the sensing residue—functional groups on the upper surface of the protective organic molecular layer, e.g., mercapto groups when the secondary polymer layer is formed upon polymerization of an alkoxysilane such as MPTMS, or a tertiary layer comprising an alkoxysilane such as MPTMS is covalently linked to the secondary polymer layer; or when further comprising a layer of multifunctional organic molecules capable of binding said target molecule via a functional group thereof, wherein said layer of multifunctional organic molecules is linked to said protective organic molecular layer. The device of the present invention may also be used for the detection of an active site-containing protein or a ligand thereof in a solution, provided that said device further comprises said ligand or active site-containing protein linked to said protective organic molecular layer. Said solution may be an aqueous solution such as a physiological solution, a bodily fluid, e.g., amniotic fluid, aqueous humour, vitreous humour, bile, blood serum, breast milk, cerebrospinal fluid, cerumen (earwax), endolymph, perilymph, female ejaculate, gastric juice, mucus, peritoneal fluid, saliva, sebum (skin oil), semen, sweat, tears, vaginal secretion, vomit and urine, or a bodily fluid-based solution, i.e., an aqueous solution in which a bodily fluid is dissolved.

In certain embodiments, the semiconductor device of the invention is configured as the MOCSER and used for the detection of a target molecule in a solution, said device further comprising a layer of multifunctional organic molecules capable of binding said target molecule via a functional group thereof, wherein said layer of multifunctional organic molecules is linked either directly or indirectly to said protective organic molecular layer, and exposure of said multifunctional organic molecules to a solution containing said target molecule causes a current change through the semiconductor device when a constant electric potential is applied between the two conducting pads.

In certain embodiments, the semiconductor device of the invention is configured as the MOCSER and used for the detection of an active site-containing protein or a ligand thereof in a solution, said device further comprising said ligand or active site-containing protein, wherein said ligand or active site-containing protein is linked either directly or indirectly to said protective organic molecular layer, and exposure of said ligand or active site-containing protein, to a solution containing said active site-containing protein or ligand, respectively, causes a current change through the semiconductor device when a constant electric potential is applied between the two conducting pads. In certain particular such embodiments, the semiconductor device is used for the detection of said active site-containing protein in said solution, wherein said device comprises said ligand linked either directly or indirectly to said protective organic molecular layer, and exposure of said ligand to a solution containing said active site-containing protein causes a current change through the device when a constant electric potential is applied between the two conducting pads. In other particular such embodiments, the semiconductor device is used for the detection of said ligand in said solution, wherein said device comprises said active site-containing protein linked either directly or indirectly to said protective organic molecular layer, and exposure of said active site-containing protein to a solution containing said ligand causes a current change through the device when a constant electric potential is applied between the two conducting pads.

The term “active site-containing protein”, as used herein, refers to a non-structural protein including, e.g., an antibody, protein antigen, enzyme, protein substrate or inhibitor, receptor, and lectin. The term “ligand”, as used herein with respect to said active site-containing protein, refers to an ion, molecule, or molecular group that binds to said active site-containing protein as defined above to form a larger complex. Non-limiting examples of active site-containing protein-ligand pairs include an antibody and its antigen, respectively, or vice versa; an enzyme and either a substrate or inhibitor thereof, respectively, of vice versa; a receptor and either a protein or organic molecule, respectively, or vice versa; and a lectin and a sugar.

In certain particular such embodiments, the semiconductor device of the invention is configured as the MOCSER and used for the detection of an active site-containing protein or a ligand thereof in a solution, said device further comprising said ligand or active site-containing protein, wherein said ligand or active site-containing protein is directly linked to said protective organic molecular layer via a functional group of the alkoxysilane or tetraalkyl orthosilicate forming the secondary polymer layer, e.g., an amino, mercapto, or hydroxyl group of said alkoxysilane or tetraalkyl orthosilicate.

In certain particular such embodiments, the semiconductor device of the invention is configured as the MOCSER and used for the detection of an active site-containing protein or a ligand thereof in a solution, said device further comprising said ligand or active site-containing protein, wherein said ligand or active site-containing protein is indirectly linked to said protective organic molecular layer.

In certain more particular such embodiments, said ligand or active site-containing protein is indirectly linked to said protective organic molecular layer via a mono- or bi-layer membrane comprising an amphiphilic compound or a mixture thereof, wherein said membrane is adhered to said protective organic molecular layer. Particular such embodiments are those wherein said ligand or active site-containing protein is immobilized on, i.e., adsorbed to, or incorporated into, said mono- or bi-layer membrane, e.g., by linking to particular chemical groups in said membrane that are capable of forming strong non-covalent or covalent bonds with said ligand or active site-containing protein.

The amphiphilic compound comprised within said monolayer or bilayer membrane may be a phospholipid, i.e., a lipid capable of forming a lipid bilayer, a biotinylated form thereof, or a mixture of the aforesaid. Such phospholipids may be either phosphoglycerides, i.e., glycerophospholipid, or phosphosphingolipids. Examples of phosphoglycerides include, without limiting, plasmalogens; phosphatidates, i.e., phosphatidic acids; phosphatidylethanolamines (cephalin); phosphatidylcholines (lecithin) such as egg phosphatidylcholin (EPC); phosphatidylserine; phospatidylinositol; phosphatidylinositol phosphate, i.e., phosphatidylinositol 3-phosphate, phosphatidylinositol 4-phosphate, or phosphatidylinositol 5-phosphate, phosphatidylinositol bisphosphate, and phosphatidylinositol triphosphate; glycolipids such as glyceroglycolipids, glycosphingolipids, and glycosylphosphatidylinopsitols; phosphatidyl sugars; and a biotinylated forms thereof such as dioleoyl-sn-glycero-3-phosphoethanolamine-N-(cap biotinyl) (BCPE), Biotin-Phosphatidylcholine (Cat. No. L-11B16, Echelon®), Biotin Phosphatidylinositol 3-phosphate (Cat. No. C-03B6, Echelon®), Biotin Phosphatidylinositol 4,5-bisphosphate (Cat. No. C-45B6, Echelon®), Biotinylated phosphatidylinositol 3,4,5-trisphosphate, and 1-((1-octanoyl-N′-biotinoyl-1,6-diamino hexane-2R-octanoyl)phosphatidyl)inositol-3,4,5-triphosphate, tetrasodium salt (PtdIns-(3,4,5)-P₃-biotin (sodium salt); Cayman, Chemical Item Number 10009531). Examples of phosphosphingolipids include, without limiting, ceramide phosphorylcholine, ceramide phosphorylethanolamine, ceramide phosphorylglycerol, and biotinylated forms thereof such as Biotin Sphingomyelin (Cat. No. S-400B, Echelon®).

The decision whether to link said ligand or active site-containing protein to said protective organic molecular layer via a lipid mono- or bi-layer membrane depends on the type and properties of said ligand or active site-containing protein, wherein formation of such a membrane might be preferred, e.g., in cases a membrane protein should be linked to the protective organic molecular layer as well as in order to avoid non-specific interactions of either or both of said ligand or active site-containing protein, and the analyte detected, i.e., said active site-containing protein or ligand, respectively, with said protective organic molecular layer.

In other more particular such embodiments, said ligand or active site-containing protein is indirectly linked to said protective organic molecular layer via a linker such as biotin, a biotin-like molecule, or a ligand-binding protein, e.g., Protein A, Protein G, avidin, streptavidin, and antibodies.

Biotin, also known as Vitamin H or coenzyme R, is a water-soluble B-complex vitamin (vitamin B₇) composed of a ureido (tetrahydroimidizalone) ring fused with a tetrahydrothiophene ring, wherein a valeric acid substituent is attached to one of the carbon atoms of the tetrahydrothiophene ring. The terms “biotin-like molecule” and “biotin-like residue” as used herein refer to any compound or a residue thereof, respectively, having a biotin-like structure, capable of binding to the tetrameric proteins avidin and streptavidin with a dissociation constant (K_{d}) similar to that of biotin, i.e., in the order of ˜10⁻¹⁵ M. Non-limiting examples of biotin-like molecules are diaminobiotin and desthiobiotin, as well as molecules comprising a tetrahydroimidizalone ring fused with a tetrahydrothiophene ring which is found in biotin, or analogs thereof such as those found in diaminobiotin and desthiobiotin.

The term “biotinylated form”, as used herein with respect to the alkoxysilane or tetraalkyl orthosilicate forming the secondary polymer layer of the protective organic molecular layer, or the amphiphilic compounds forming the mono- or bi-layer membrane, refers to any of said alkoxysilanes, tetraalkyl orthosilicates, or amphiphilic compounds, respectively, when covalently attached to a biotin residue or to a residue of a biotin-like molecule, e.g., via one of the functional groups thereof. Biotinylation of alkoxysilanes, tetraalkyl orthosilicates, or amphiphilic compounds as defined above can be conducted using any technology or method commonly known in the art.

Protein A is a surface protein originally found in the cell wall of Staphylococcus aureus, capable of binding immunoglobulins. The protein is composed of five homologous Ig-binding domains that fold into a three-helix bundle, wherein each domain is capable of binding proteins from many of mammalian species, preferably IgGs. In particular, Protein A binds the heavy chain with the Fc region of most immunoglobulins and also within the Fab region in the case of the human VH3 family.

Protein G is an immunoglobulin-binding protein expressed in group C and G Streptococcal bacteria much like Protein A but with different specificities. It is a cell surface protein that is commonly used in purifying antibodies through its binding to the Fc region. Protein G in its natural form also binds albumin; however, because serum albumin is a major contaminant of antibody sources, the albumin binding site has been removed from recombinant forms of Protein G.

Avidin is a homotetrameric biotin-binding protein having four identical subunits, produced in the oviducts of birds, reptiles and amphibians deposited in the whites of their eggs. Each one of the subunits can bind to biotin with high affinity and specificity, wherein the K_{d} of avidin is ˜10⁻¹⁵ M, making it one of the strongest known non-covalent bonds.

Streptavidin is a protein purified from Streptomyces avidinii. Streptavidin homo-tetramers have an extraordinarily high affinity for biotin, wherein its binding to biotin is one of the strongest non-covalent interactions known in nature.

The term “antibodies”, as used herein with respect to a ligand-binding protein, refers to polyclonal and monoclonal antibodies of avian, e.g., chicken, and mammals, including humans, and to fragments thereof such as F(ab′)₂ fragments of polyclonal antibodies, and Fab fragments and single-chain Fv fragments of monoclonal antibodies. The term also refers to chimeric, humanized and dual-specific antibodies.

Ligand binding proteins such as Protein A and Protein G can be used, e.g., when the active site-containing protein indirectly linked to the protective organic molecular layer is an antibody. Ligand binding proteins such as streptavidin and avidin can be used, e.g., to bind biotin or a biotin-like molecule, to which said ligand or active site-containing protein is linked. An antibody can be used as a ligand binding protein, e.g., when the active site-containing protein indirectly linked to the protective organic molecular layer is an antigen capable of forming strong interactions with said antibody.

In certain specific embodiments, the semiconductor device of the invention is configured as the MOCSER and used for the detection of an active site-containing protein or a ligand thereof in a solution, said device further comprising said ligand or active site-containing protein indirectly linked to said protective organic molecular layer via a mono- or bi-layer membrane comprising a mixture of an amphiphilic compound and a biotinylated form of an amphiphilic compound, wherein a biotinylated form of said ligand or active site-containing protein is non-covalently attached via an avidin or streptavidin molecule to the biotin or biotin-like residues in said mono- or bi-layer membrane.

In other specific embodiments, the semiconductor device of the invention is configured as the MOCSER and used for the detection of an active site-containing protein or a ligand thereof in a solution, said device further comprising said ligand or active site-containing protein indirectly linked to said protective organic molecular layer via biotin or a biotin-like molecule, wherein said biotin or biotin-like molecule is covalently linked to a functional group in said protective organic molecular layer, and a biotinylated form of said ligand or active site-containing protein is non-covalently attached via an avidin or streptavidin molecule to the biotin or biotin-like residues linked to said protective organic molecular layer.

In further specific embodiments such as that exemplified in Study 2, the semiconductor device of the invention is configured as the MOCSER and used for the detection of an active site-containing protein or a ligand thereof in a solution, said device further comprising said ligand or active site-containing protein indirectly linked to said protective organic molecular layer via a ligand binding protein such as Protein A, Protein G, streptavidin, avidin or an antibody, wherein said ligand binding protein is covalently linked to a functional group in said protective organic molecular layer, and non-covalently attached to said ligand or active site-containing protein.

Semiconductor devices according to the present invention, when configured as the MOCSER, may be used for the detection of either a target molecule, or an active site-containing protein or a ligand thereof, in a solution, as described above. Moreover, such devices may be used for quantification of the analyte detected, i.e., said target molecule, active site-containing protein or ligand thereof, in said solution, wherein the current change through the semiconductor device when a constant electric potential is applied between the two conducting pads is proportional to the concentration of said analyte in the solution.

In another aspect, the present invention thus relates to a method for the detection of a target molecule in a solution, said method comprising (i) exposing a semiconductor device as defined above, when configured as a MOCSER, to said solution; and (ii) monitoring the presence of said target molecules in said solution according to the changes in the current measured in said semiconductor device when a constant electric potential is applied between the two conducting pads. According to this method, exposure of functional groups in said protective organic molecular layer, more particularly functional groups of the alkoxysilane or tetraalkyl orthosilicate forming the secondary polymer layer or said tertiary layer deposited on top of said secondary polymer layer, if present, to a solution containing said target molecule causes a current change through the semiconductor device when a constant electric potential is applied between the two conducting pads.

In one embodiment, said target molecule is ammonia; said secondary polymer layer is obtained upon polymerization under basic conditions of an alkoxysilane of the formula HS—R₁—Si(OR₂)₃ such as MPTMS, or said tertiary layer, if present, comprises an alkoxysilane of the formula HS—R₁—Si(OR₂)₃; and exposure of the mercapto groups of said alkoxysilane to a solution containing ammonia causes a current change through the semiconductor device when a constant electric potential is applied between the two conducting pads.

In a further aspect, the present invention relates to a method for the detection of a target molecule in a solution, said method comprising: (i) exposing a semiconductor device as defined above, when configured as a MOCSER and further comprises a layer of multifunctional organic molecules capable of binding said target molecule via a functional group thereof, to said solution; and (ii) monitoring the presence of said target molecules in said solution according to the changes in the current measured in said semiconductor device when a constant electric potential is applied between the two conducting pads. According to this method, exposure of said multifunctional organic molecules to a solution containing said target molecule causes a current change through the semiconductor device when a constant electric potential is applied between the two conducting pads.

In certain embodiments, the methods described above are further used for quantification of said target molecule in said solution, wherein the current change is proportional to the concentration of said target molecule in said solution.

In yet another aspect, the present invention relates to a method for the detection of an active site-containing protein or a ligand thereof in a solution, said method comprising: (i) exposing a semiconductor device as defined above, when configured as a MOCSER and further comprises said ligand or active site-containing protein, to said solution; and (ii) monitoring the presence of said active site-containing protein or ligand in said solution according to the changes in the current measured in said semiconductor device when a constant electric potential is applied between the two conducting pads. As exemplified in Study 2, this method can be used, e.g., for the detection of hemoglobin in a solution, wherein said active site-containing protein is hemoglobin, said ligand is a hemoglobin antibody, and said hemoglobin antibody is linked either directly or indirectly to said protective organic molecular layer.

In certain embodiments, this method is further used for quantification of said active site-containing protein or ligand thereof in said solution, wherein the current change is proportional to the concentration of said active site-containing protein or ligand thereof in said solution. In other embodiments, this method is used for studying receptor-ligand pair interactions, more particularly, for monitoring the interaction of a receptor in a solution with a ligand directly or indirectly linked, as defined above, to the protective organic molecular layer, or vice versa.

The invention will now be illustrated by the following non-limiting Examples.

### EXAMPLES

### Experimental

### Materials

3-Mercaptopropyl trimethoxysilane (MPTMS) was purchased from Sigma. Sodium phosphate monobasic (Cat. No. 567545) and sodium phosphate dibasic (Cat. No. 567550) were obtained from Merck KGaA, Darmstadt, Germany. Polydimethylsiloxane (PDMS) was purchased from DowCorning, Inc. Med-1000 silicon adhesive was obtained from NuSil Silicon Technology. Deionized Milli Q water (DDW) was used for the buffer preparation and experiments. GaAs Pseudomorphic High Electron Mobility Transistor (pHEMT) wafers for MOCSER devices fabrication were supplied by IQE, Inc.

### Polymer Deposition

A comparison between the different methods for polymer deposition is presented in Table 1.

### The Standard Procedure

The procedure for depositing a protective layer of MPTMS on GaAs-based devices was previously reported (Bavli et al., 2012; Tatikonda et al., 2013). According to this “standard” procedure, GaAs substrates are first cleaned in isopropanol, acetone and ethanol (EtOH) for 10 minutes each, and are then oxidized by UVOCS for 10 minutes. Following oxidation, the samples are etched for 5 seconds in HF 2%, rinsed in DDW, etched for 30 seconds in NH₄OH 25%, and finally rinsed in DDW again. After etching, the substrates are dried with nitrogen and immediately immersed in ethanol solutions of MPTMS; in previous works, concentration of either 0.3 vol. % (16 mM) (Bavli et al., 2012) or 0.4 vol. % (21.5 mM) (Tatikonda et al., 2013) were used. Placing in a water bath at 50° C. for 4 hours allows primary MPTMS layer adsorption with thiol binding to the substrate and with the reactive methoxy groups pointing outwards (Hou et al., 1997). Next, polymerization of MPTMS is initiated by adding NH₄OH 25% (3 vol. % NH₄OH for 0.3 vol. % MPTMS concentration, and 4 vol. % NH₄OH for 0.4 vol. % MPTMS concentration). The solution is kept at 50° C. for additional 16 hours, and the samples are then rinsed with ethanol and dried under a stream of nitrogen (Table 1, Standard procedure).

### The New Procedure

The new procedure completely separates the two steps of the standard procedure, i.e., the deposition of the primary layer of MPTMS and the polymerization stage. In particular, after the GaAs substrates have been cleaned, oxidized, and etched as in the standard procedure, they immediately immersed in a solution of 0.1 vol. % (5.3 mM) MPTMS in ethanol. Next, the samples are placed in a water bath at 50° C. for 8 hours for primary layer deposition. For the polymerization stage, the MPTMS concentration is increased to either 0.3 or 0.4 vol. % by adding MPTMS to the first step solution, and NH₄OH 25% is added to initiate the polymerization (3 vol. % NH₄OH for 0.3 vol. % MPTMS concentration, and 4 vol. % NH₄OH for 0.4 vol. % MPTMS concentration). The samples are returned to the bath at 50° C. for additional 16 hours, and are then rinsed with ethanol and dried with nitrogen (Table 1, New procedure).

A schematic representation of the new procedure is shown in Scheme 1, wherein in the first stage, MPTMS primary layer is adsorbed in low concentration solution, while during the polymerization stage, higher concentration of MPTMS is used with added condensation agent NH₄OH.

Another variation of the second stage includes preparing the new solution for the polymerization with 0.3 or 0.4 vol. % of MPTMS concentration. For better dispersion of the condensation agent in the solution, NH₄OH is added to the MPTMS solution before immersing the sample, and the mixture is thoroughly shaken with Vortex for 30 seconds. Here, the samples are immediately transferred from the vial with the “first stage” solution to the polymerization solution. The samples are then placed in a bath at 50° C. for 16 hours, and are then rinsed with ethanol and dried with nitrogen.

TABLE 1
Step-by-step MPTMS polymer deposition standard vs. new procedure

| | | |
|---|---|---|
| | | New procedure |
| Standard procedure | New procedure | with NH₄OH dispersion |

Clean in isopropanol, acetone and ethanol (10 min each); UVOCS for 10 min; Etch in
2% HF (5 s), rinse in DDW (1 s), etch in 25% NH₄OH (30 s), rinse in DDW (1 s).

| Immerse in MPTMS | Immerse in MPTMS solution: 0.1 vol. % solution of | |
|---|---|---|
| solution: 0.3 vol. % or 0.4 | MPTMS in EtOH - 8 hours at 50° C. | |
| vol. % solution of MPTMS | | |
| in EtOH - 4 hours at 50° C. | | |
| Add NH₄OH (3 vol. % | 1. Increase concentration of | 1. New solution for |
| NH₄OH for 0.3 vol. % | MPTMS in the adsorption | polymerization stage - 0.3 |
| MPTMS concentration, 4 | solution to 0.3 vol. % (add | vol. % MPTMS in EtOH. |
| vol. % NH₄OH for 0.4 vol. % | MPTMS dissolved in EtOH | 2. Add NH₄OH (3 vol. %), |
| MPTMS) | to get overall 0.3 vol. %) | mix by shaking with Vortex |
| | 2. Add NH₄OH (3 vol. %) | (30 s) |
| | | 3. Transfer immediately the |
| | | sample from 0.1 vol. % |
| | | solution to the new solution |

Polymerization - 16 hours at 50° C.
Rinse samples with ethanol and dry with N₂

In order to estimate the thickness of the polymer, GaAs samples were coated with MPTMS film in parallel with MOCSER devices and the film thickness was measured by ellipsometry (J. A. Woollam, model M-2000V) immediately after the polymer deposition.

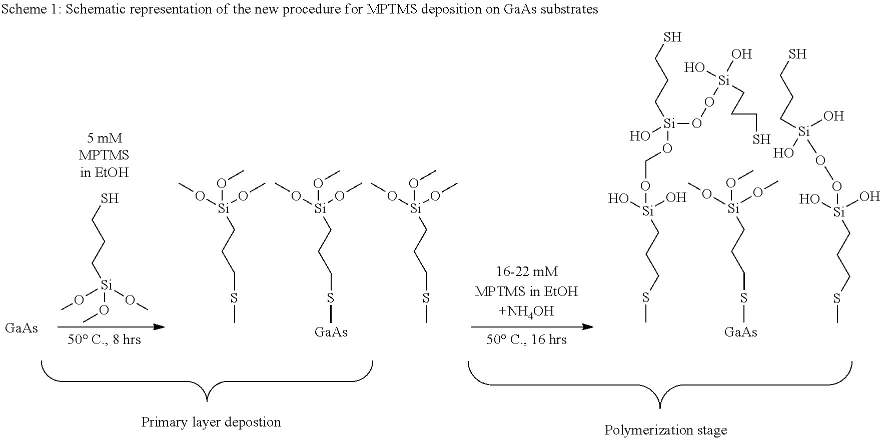

### AFM Imaging

The quality of both the primary MPTMS layer and the MPTMS polymer layer adsorbed on the GaAs substrates and the MOCSER devices was evaluated by AFM imaging. The topography images of MPTMS coatings on GaAs were acquired using the AFM P47 (NT-MDT, Zelenograd) equipped with a small scanner. Images were recorded in the tapping mode in the air at the room temperature (22-24° C.) using silicon micro cantilevers (OMCL-AC240TS-W2, Olympus) with a nominal spring constant of 2 N/m and a resonant frequency of 70 kHz (manufacturer specified). The set point ratio was adjusted to 0.75-0.8 (corresponding to the “light” tapping) and the scan rate was set to 1 Hz. Imaging was carried out at different scales (13×13, 5×5 and 3×3 μm) to verify the consistency and robustness of the evaluated structures. Image analysis was performed using Nova 1.0.26.1443 software.

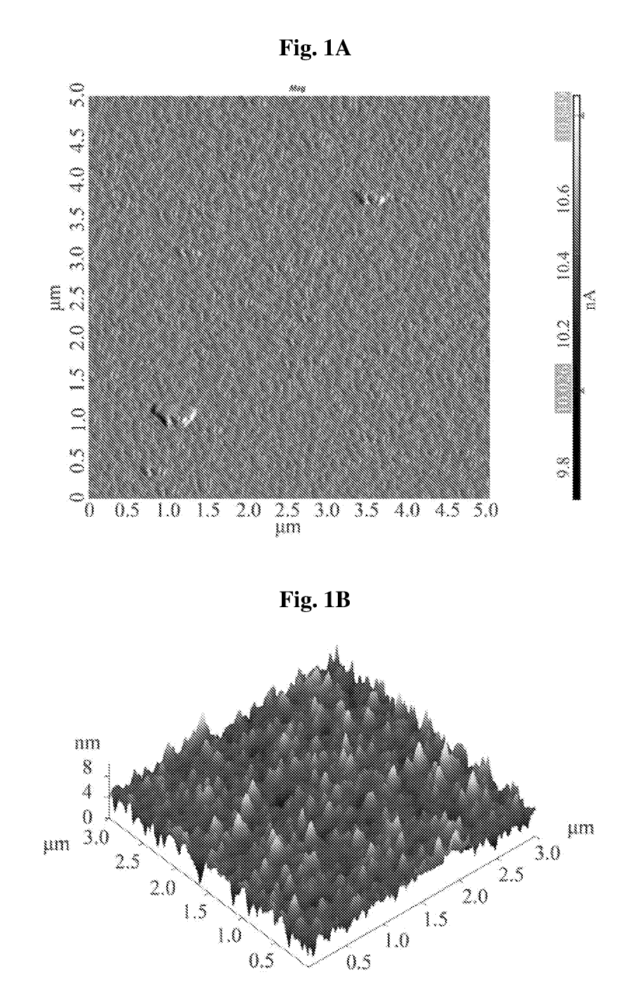

The AFM characterization of the obtained MPTMS layers was performed both on n-doped GaAs substrates and on MOCSER devices. In order to verify that the MPTMS polymer prepared on the devices is similar to the polymer adsorbed on the GaAs substrates, MOCSER devices and GaAs-based samples were coated with MPTMS under the same conditions and the surfaces were then characterized with AFM. Indeed, the polymer layers adsorbed under the same conditions on GaAs and MOCSER devices produced similar AFM data (FIG. 1).

As shown in FIGS. 1A and 1C, there are always surface defects present that look like polymer agglomerations on top of the polymer layer. In order to estimate the overall surface quality, large areas of 13×13 or 5×5 μm were scanned. For quantitative surface roughness analysis we chose defect-free regions, so that several regions of 3×3 μm were analyzed for each sample.

### Device Fabrication and Electrical Measurements

GaAs/AlGaAs MOCSER devices with a 600-μm long and 200-μm wide conducting channel were fabricated by standard photolithography techniques based on GaAs pHEMT structures. Each die contained 16 devices that were measured simultaneously. In the data analysis, only devices exhibiting normal current-voltage characteristics at the beginning of the measurement were included. All the electrical measurements were performed on wire-bonded devices using two Keithley 236 source-measure units. The system was controlled and monitored by a Labview application (version 8.6). A voltage of 1.0 V was applied between the source and drain of the MOCSER devices, and the change in source-drain current was monitored as a function of time.

The measured current on each MOCSER was normalized according to the equation I=(I−I₀)×10³/I₀, where I₀ is the baseline current with pH 7, before introducing buffer with pH 6. In addition, in order to remove the influence of current drift with time, the baseline correction was performed according to baseline current with pH 7, and the average signal on the working devices was calculated.

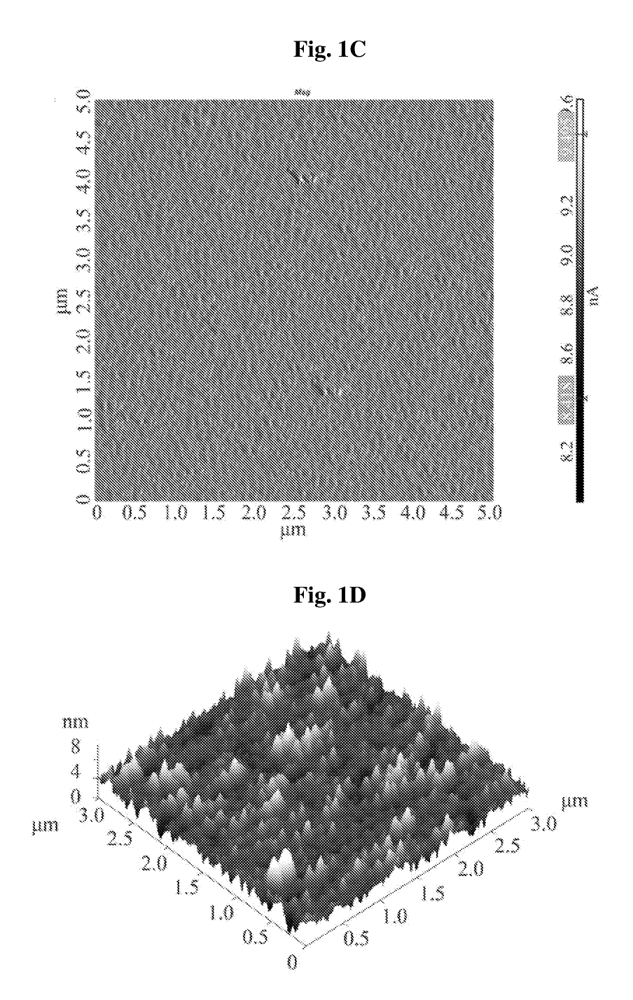

A polydimethylsiloxane (PDMS)-based flow cell (4 mm in length and width and 0.6 mm in height) was fixed on top of the sensing area of the MOCSER with MED-1000 silicon adhesive. Transferring of analytes and buffer solutions to the MOCSER devices was performed at 0.03 ml/min using a peristaltic pump (EP-1 Econo pump, Bio-Rad Laboratories Israel) with teflon pipes (inner diameter of 0.8 mm). Ag/AgCl reference electrode connected via a salt bridge to the sensing chamber was used to provide a stable reference potential in the solution (FIG. 2).

pH measurements were used (Bavli et al., 2012) to estimate the sensitivity and stability of the polymer-coated devices. The change in the current was recorded upon exposure to phosphate buffer solutions (0.05 M) with pH 6.0, 7.0 and 8.0, for 1000 s each. Initially the measurements were performed on freshly prepared devices; and the system was then left overnight to measure the baseline current at pH 7.0. The experiment was repeated after 7-15 hours of continuous electrical operation.

### Additional Results

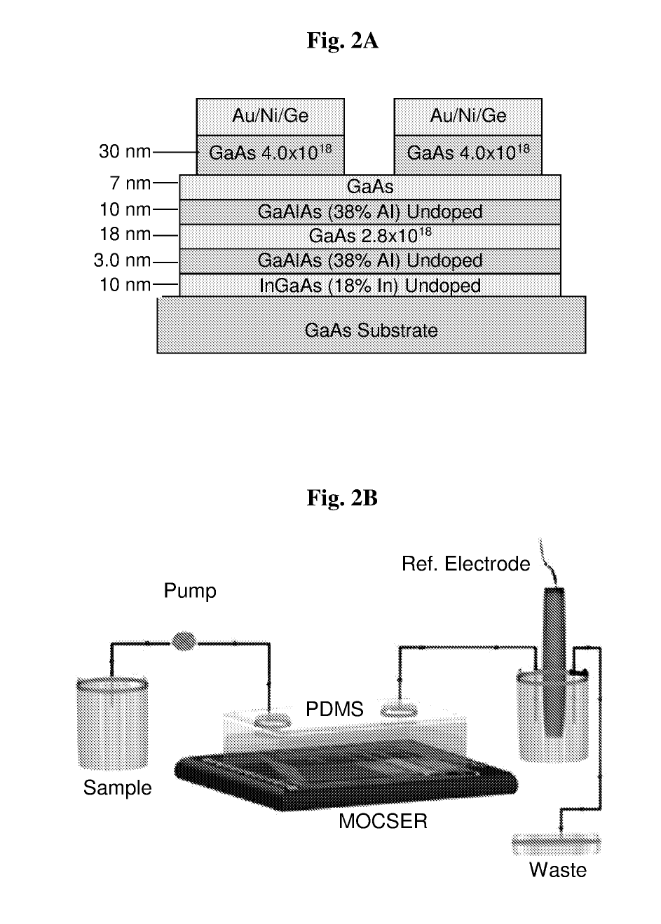

While trying to evaluate optimal conditions for polymerization, it has been found that solutions with low concentrations of MPTMS (0.1 and 0.2 vol. % MPTMS in EtOH) do not allow formation of a continuous polymerized layer. As shown in FIG. 3, the structures obtained in these cases look like a continuous primary layer with circular polymer agglomerations on top of it.

### Study 1. MOCSER Devices Protected According to the Procedure Disclosed Herein are Highly Stable and Sensitive in Aqueous Solutions

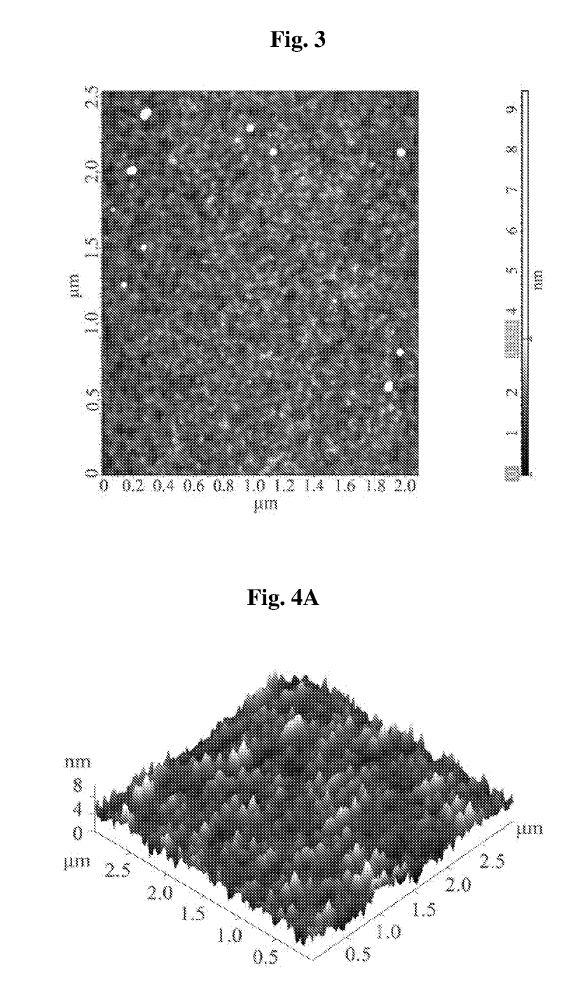

When an MPTMS layer is deposited by the standard procedure, the GaAs substrate exhibits good corrosion stability when exposed to an aqueous environment for up to 24 hours (Bavli et al., 2012; Kirchner et al., 2002). However, continuous electrical load causes fast device degradation, expressed in reduced sensitivity and even device failure after few hours of operation. The degradation of the devices occurs apparently due to increase in temperature that leads to growing amount of defects in the MPTMS coating, penetration of water molecules to GaAs-polymer interface, and subsequent etching of the surface of the device. Characterization by AFM shows that after using the device at 1 V for a few hours, the polymer surface roughness (peak-to-peak and RMS values) increases relatively to freshly prepared device (FIG. 4).

We aimed to produce MPTMS polymer layer strongly bound to the substrate with low-defect surface for effective long-term protection of GaAs-based devices operating in biological conditions. As previously shown, adsorption behavior of silane coupling agents depends strongly on the solution concentration, wherein at a low concentration the molecules are adsorbed in a more regular fashion than in the case of a high concentration (Nishiyama et al., 1989). Another important parameter is the deposition time. The strategy for forming effective protective film on GaAs substrates was thus separating the two processes occurring in MPTMS deposition, using low concentration of the adsorption solution and increased deposition time during the primary layer formation, while using regular concentrations and deposition times for the polymerization step. This procedure allows self-organization of the adsorbed molecules on the GaAs surface during the first step, resulting in dense high-quality primary layer.

In order to evaluate the new coating procedure, we probed, by ellipsometry and AFM, the primary layer adsorbed under different conditions and the surface of the polymerized MPTMS layer. The characterization was performed both on an n-doped GaAs substrate and on MOCSER devices.

### Primary MPTMS Layer Characterization

For probing the influence of the adsorption conditions on the quality of the MPTMS primary layer, we prepared a set of films on GaAs; in these experiments both the MPTMS concentrations and adsorption time were varied, and no NH₄OH was added. No polymerization occurs in this case, thus only a primary layer is deposited. The AFM and ellipsometry characterization data are summarized in Table 2.

Adsorption of MPTMS on GaAs from ethanol solutions with different concentrations results in multilayer film more than 2 nm thick. This can be attributed to oligomerization of MPTMS in solution and the subsequent adsorption on the GaAs surface. If the film deposited for 4 hours, it is not evident from the AFM results that the quality of the primary layer depends on the concentration of the adsorption solution (changes in RMS values are within error range). However, we found it to depend strongly on the deposition time. For samples prepared from 0.4 vol. % MPTMS solutions, increasing the deposition time from 4 to 20 hours results in lower peak-to-peak values and decreasing of the RMS values from 0.60 to 0.39 nm. In case of 0.1 vol. % MPTMS solutions, RMS improves from 0.55 nm for 4 hours to 0.35 nm for 8 hours of adsorption, while further increasing of deposition time does not affect the surface smoothness. Moreover, in case of 8 hours deposition, decreasing concentration of the adsorption solution to 0.1 vol. % MPTMS significantly improves the roughness (RMS=0.35 nm) relatively to high concentration of 0.4 vol. % (RMS=0.45 nm).

Thus, setting the primary layer conditions to 0.1 vol. % MPTMS and 8 hours of deposition produces a smooth primary layer with better adhesion to the substrate, leading to a more uniform polymer layer produced during the second stage of the new procedure.

TABLE 2
Summary of the primary MPTMS layer adsorption experiments

| MPTMS | | | | |
|---|---|---|---|---|
| concentration, | Deposition | | | |
| vol. % | time | Thickness | Peak-to-peak | RMS |
| MPTMS in EtOH | (hour) | (nm) | (nm) | (nm) |
| 0.4 | 4 | 3.6 ± 0.6 | 5.3 ± 0 | 0.60 ± 0.01 |
| 0.4 | 8 | 4.9 ± 0.8 | 9.1 ± 5.9 | 0.45 ± 0.21 |
| 0.4 | 20 | 5.0 ± 1.0 | 4.6 ± 1.4 | 0.39 ± 0.05 |
| 0.2* | 4 | 3.1 ± 0.04 | 5.0 ± 0.3 | 0.58 ± 0.03 |
| 0.1 | 4 | 2.4 ± 0.3 | 4.7 ± 0.4 | 0.55 ± 0.05 |
| 0.1 | 8 | 3.5 ± 0.3 | 3.5 ± 0.1 | 0.35 ± 0.01 |
| 0.1 | 20 | 3.6 ± 0.3 | 3.2 ± 0.3 | 0.35 ± 0.02 |

*Only a single sample was measured.

### Characterization of the Polymerized MPTMS Layer

As found, the primary layer deposition conditions significantly affect the polymerization. More specifically, solutions with low concentrations of MPTMS (0.1 and 0.2 vol. % MPTMS in EtOH) do not form a continuous polymerized layer, whereas solutions with higher concentrations of 0.3 and 0.4 vol. % of MPTMS result in a continuous polymer layer.

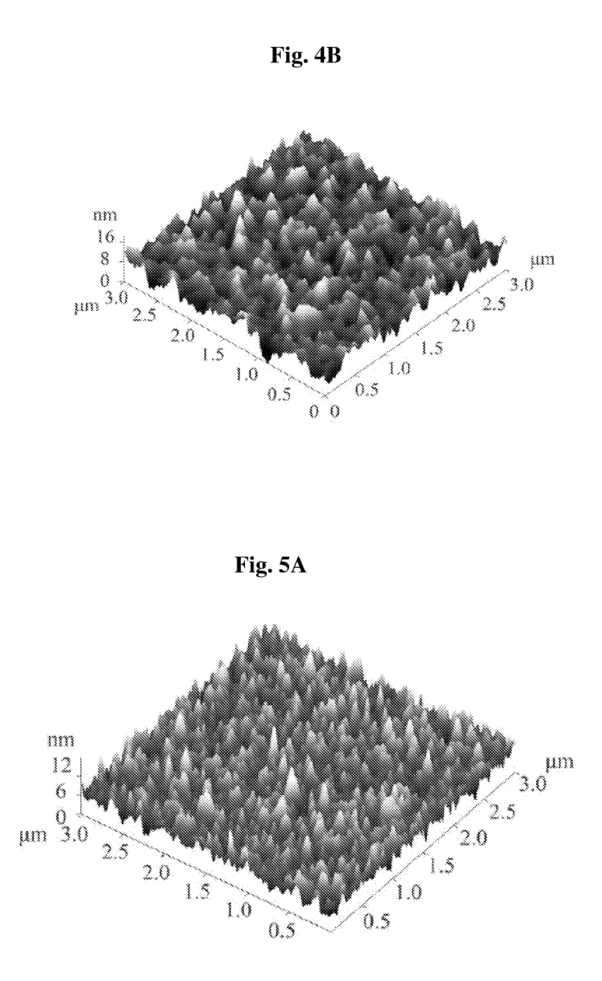

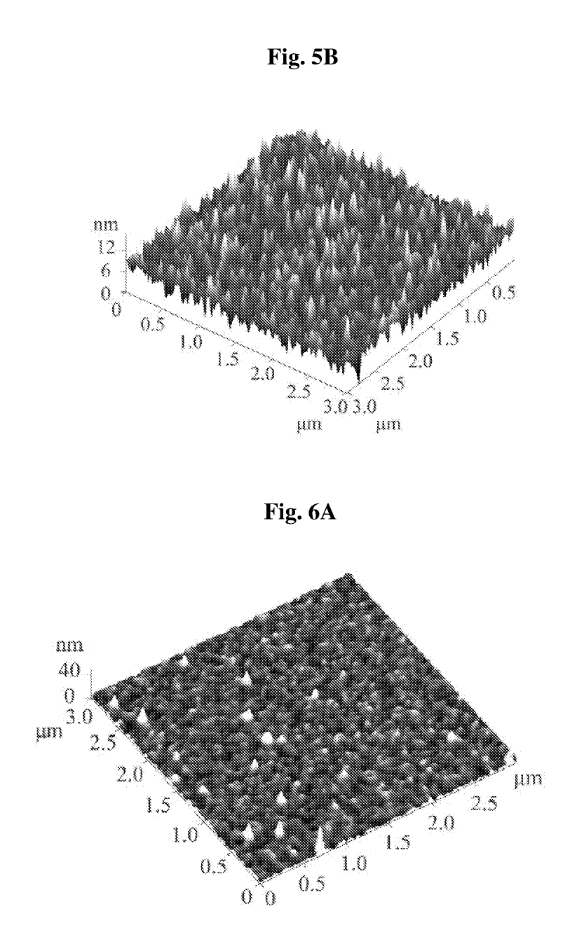

Thus, we used 0.3 and 0.4 vol. % MPTMS concentrations for the polymerization stage, while varying the conditions for the primary layer adsorption. This included increasing the deposition time from 4 to 8 hours and decreasing the concentration to 0.1 vol. % MPTMS during the first step. Several samples were prepared on different days and the thickness of the resulting polymer was estimated by ellipsometry. The high deviations in the MPTMS layer thickness observed (Table 3) are attributed to variations in air humidity during sample preparation. As a consequence, comparative roughness analysis is problematic for polymer samples prepared on different days. For this reason, we compared the AFM data of samples prepared on the same day for concentrations of 0.4 vol. % MPTMS (Set #1), and 0.3 vol. % MPTMS (Set #2). Another difficulty in obtaining quantitative roughness estimation arises from the defects present on the polymer surface. To reduce errors resulting from polymer agglomerations, we performed roughness analysis on defect-free areas. Typical AFM images are shown in FIGS. 5 and 6; and the AFM data analysis and ellipsometry characterization are summarized in Table 3.

When the polymer deposition is performed in a solution of 0.4 vol. % MPTMS, the thickness of the resulting layer ranges from 18 to 33 nm, whereas for 0.3 vol. % MPTMS the polymer thickness decreases to 15-29 nm. Changing the primary layer adsorption conditions does not affect the quality of the polymer layer with the 0.4 vol. % MPTMS concentration. In contrast, for samples prepared in a 0.3 vol. % MPTMS solution, the primary adsorbed layer profoundly affects the overall polymer quality. When the first layer is adsorbed from solutions with low concentrations and long deposition times, the peak-to-peak and RMS values in the AFM images are significantly reduced, indicating the role of the primary layer in improving the surface quality.

TABLE 3
Summary of the MPTMS polymer deposition experiments

| | Primary layer conditions | | Polymer layer conditions | | | |
|---|---|---|---|---|---|---|
| | MPTMS concentration, | | MPTMS concentration, | | 3 × 3 scan | |
| Set | vol. % MPTMS | Deposition | vol. % MPTMS | Thickness | Peak-to- | RMS |
| # | in EtOH | time (hour) | in EtOH | (nm) | peak (nm) | (nm) |
| 1 | 0.4 | 8 | 0.4 | 24.9 ± 7.1 | 19.5 ± 2.5 | 2.3 ± 0.1 |
| 1 | 0.1 | 8 | 0.4 | 26.1 ± 7.0 | 19.3 ± 0.7 | 2.3 ± 0.1 |
| 2 | 0.3 | 4 | 0.3 | 19.9 ± 2.7 | 23.0 ± 7.5 | 1.9 ± 0.2 |
| 2 | 0.1 | 8 | 0.3 | 22.3 ± 6.7 | 16.7 ± 4.5 | 1.5 ± 0.1 |
| 2 | 0.1 | 8 | 0.3, with NH₄OH | 15.9 ± 1.2 | 15.0 ± 0.1 | 1.9 ± 0.01 |
| | | | dispersion | | | |

To test the influence of the condensation agent dispersion in the polymerization solution, we prepared a set of samples in which the MPTMS solution for the second stage was thoroughly mixed with NH₄OH by shaking with Vortex before transferring the sample from the first-step solution. Samples polymerized in 0.4 vol. % MPTMS solutions resulted in a non-uniform polymer surface 30-40 nm thick. In contrast, with 0.3 vol. % MPTMS, this treatment exhibited a significantly lower thickness of the polymer than in samples prepared without shaking. The roughness of the surface was improved in terms of peak-to-peak values, but RMS values remained essentially the same as in the standard procedure with a 0.3 vol. % MPTMS concentration (see Table 3).

### Sensing Measurements

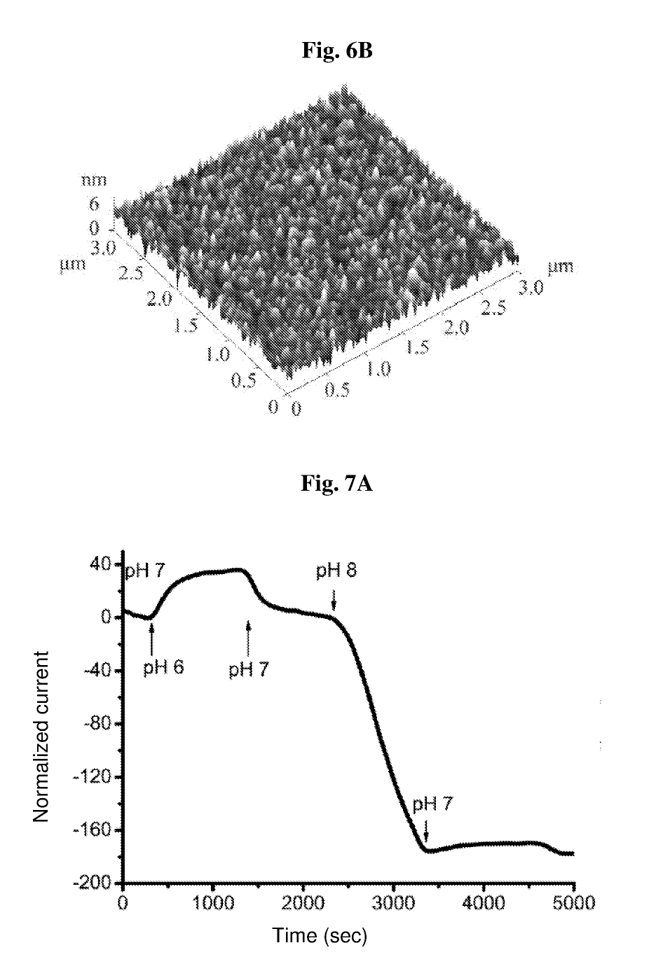

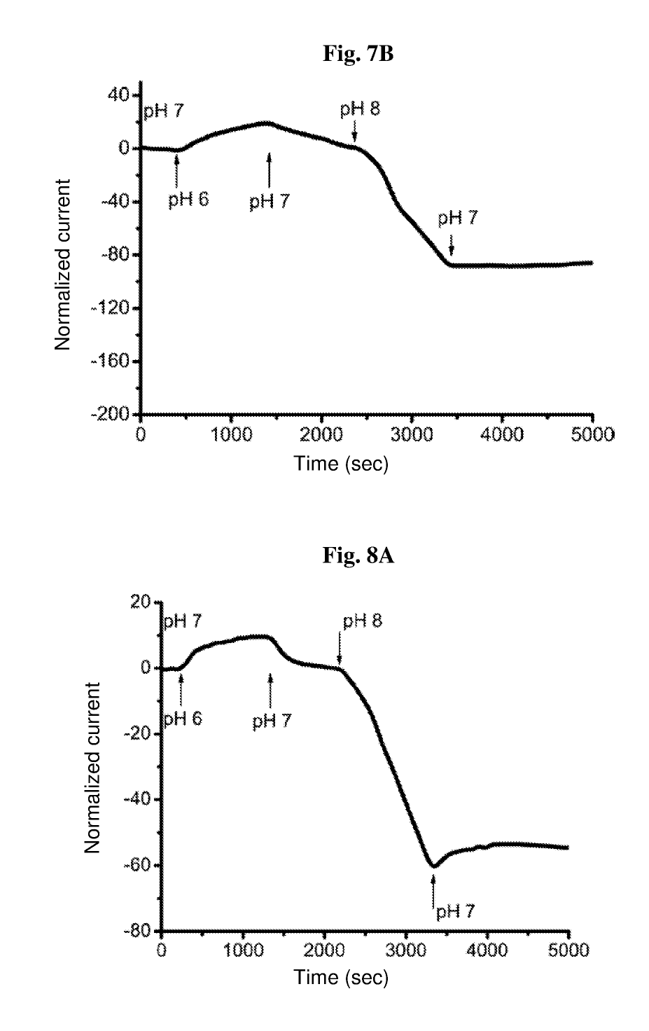

The ultimate check for assessing the quality of the protection layer lies in the sensing measurements, which provide information on both the sensitivity and the ability of the layer to protect the device for a long time, i.e., the stability. To this end, we prepared several MOCSER devices coated with MPTMS under different conditions, wherein on each one of the devices a series of measurements were performed, to test their stability and sensitivity. Phosphate buffer solutions at different pHs were used. The change in the normalized current as a function of time upon exposure to solutions with pH 6, 7 and 8 is shown in FIGS. 7 and 8. The measurements were conducted first on freshly prepared devices and were then repeated after 7-15 hours of continuous operation of the device. The results of sensing experiments are summarized in Table 4.

In general, devices coated with a polymer prepared in 0.4 vol. % MPTMS solutions (Table 4, cases a and b) are significantly less stable than devices prepared in 0.3 vol. % MPTMS solutions. Some of the MOCSER devices, prepared with the 0.4 vol. % MPTMS solution, stopped working already during the initial pH measurements, and those that survived after 12 hours lost their sensitivity. In case of the modified primary layer (0.1 vol. % MPTMS for 8 hours) and the 0.4 vol. % MPTMS solution for the polymerization step (Table 4, case c), the devices are sensitive to pH changes both when fresh and after 12 hours of measurements. Moreover, the sensitivity of the devices prepared with 0.4 vol. % MPTMS solution during polymerization is lower than that observed in case of 0.3 vol. % MPTMS polymerization, apparently due to higher thickness of the protective layer.

MOCSER device prepared with a 0.3 vol. % MPTMS solution according to the standard procedure (Table 4, case d) exhibits high sensitivity when fresh, but only 6 out of 10 MOCSER channels survived the 7-hour measurements and the device sensitivity significantly decreases (FIG. 7). After 15 hours of continuous measurements, most of the devices fail.

When the device is coated with MPTMS according to the new procedure (Table 4, case e), all 8 channels that worked at the beginning of the experiment exhibit stable performance. Interestingly, the same 8 channels were still sensitive to pH changes after 15 hours of continuous measurements. FIG. 8 shows the average signal obtained from the 8 MOCSER channels upon changes in pH with a fresh MPTMS coating (8A) and after 15 hours of operation (8B).

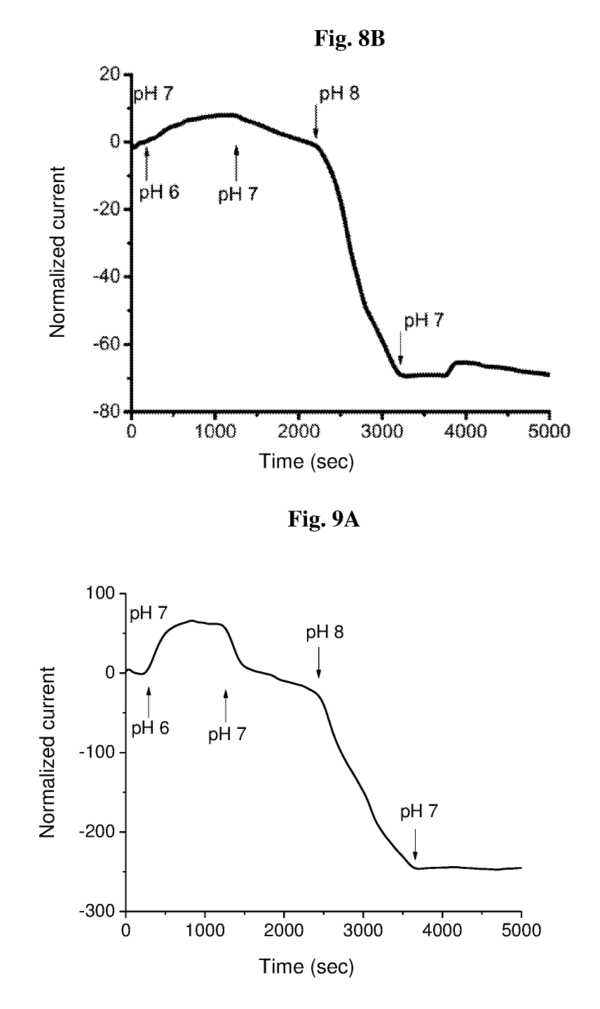

We also tested the stability and sensitivity of the MOCSER device when MPTMS is polymerized in a solution with NH₄OH dispersed by Vortex (Table 4, case f). Here the sensitivity is significantly higher, due to the thinner polymer layer obtained (FIG. 9). Nine out of ten operating channels were still sensitive after 7 hours of measurements. After 15 hours of measurements only 6 MOCSER channels functioned properly; however, with reduced sensitivity (which is still comparable to case e, data not shown).

TABLE 4
Summary of electrical measurements

| | Primary layer conditions | | Polymer layer conditions | | | |
|---|---|---|---|---|---|---|
| | MPTMS concentration | | MPTMS concentration | | | |
| Device | (vol. % MPTMS | Deposition | (vol. % MPTMS | Thickness | | |
| type | in EtOH) | time (hour) | in EtOH) | (nm) | Stability | Sensitivity |
| a | 0.4 | 4 | 0.4 | 22.3 ± 2.9 | − | − |
| b | 0.4 | 8 | 0.4 | 24.9 ± 7.1 | − | ± |
| c | 0.1 | 8 | 0.4 | 26.1 ± 7.0 | ± | + |
| d | 0.3 | 4 | 0.3 | 19.9 ± 2.7 | ± | + |
| e | 0.1 | 8 | 0.3 | 22.3 ± 6.7 | + | + |
| f | 0.1 | 8 | 0.3, Mixture shaken | 15.9 ± 1.2 | ± | ++ |
| | | | with NH₄OH | | | |

Following the sensing experiments, the effect of the electrical measurements on the surface of the protecting polymer was probed by AFM. We characterized the surfaces of the devices prepared in 0.3 vol. % MPTMS polymerization solutions. First, a scan of 13×13 μm was performed followed by measuring several regions of 3×3 μm. The AFM data are summarized in Table 5.

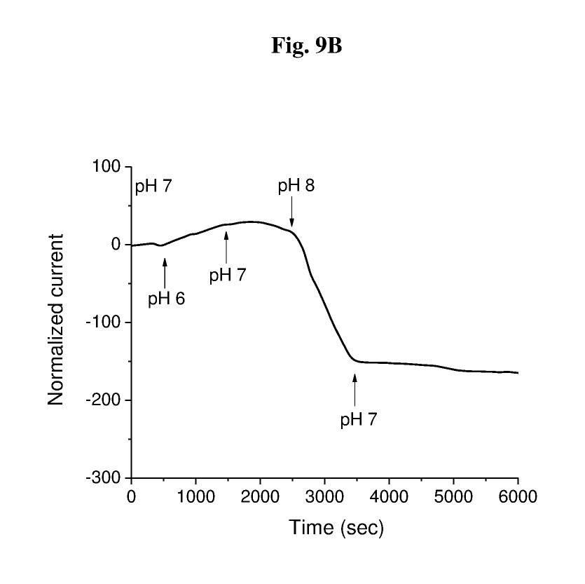

In the case of devices prepared by the standard procedure (Table 4, case d), it is obvious that the number of surface defects significantly increases after the electrical measurements (FIGS. 10A-10B). Roughness analysis reveals that not only the density of defects increased but also the peak-to-peak and RMS values increased dramatically (Table 5).

When MOCSER devices are coated with MPTMS according to the new procedure (adsorption of primary layer in 0.1 vol. % MPTMS solution for 8 hours, and 0.3 vol. % MPTMS solution for polymerization, Table 4, case e), a significantly lower number of surface defects is observed on an MPTMS-coated surface of the fresh samples, both for the normal addition of NH₄OH (FIG. 10C) and for the high-dispersion NH₄OH variation (data not shown). Although the number of defects increases after electrical test in these two cases, the number of surface defects is still much lower than that observed for devices coated according to the standard procedure (FIG. 10D). Moreover, increase in surface roughness during the device operation is much less significant than in the case of the standard MPTMS deposition: peak-to-peak and RMS values of the operated device are comparable to those of fresh device prepared by the old method.

TABLE 5
AFM data for samples and devices prepared with 0.3 vol. % MPTMS solution
in EtOH for polymer layers before and after electrical measurements

| Primary layer conditions | | | | | |
|---|---|---|---|---|---|
| MPTMS | | Polymerized | | | |
| concentration (vol. % | Deposition | layer | Sample | Peak-to-peak | RMS |
| MPTMS in EtOH) | time (hour) | conditions | type | (nm) | (nm) |
| 0.3 | 4 | Normal | Fresh | 58.2 ± 5.2 | 2.4 ± 0.3 |
| | | NH₄OH | samples | | |
| | | addition | | | |
| | | | Measured | 94.1 ± 49.7 | 5.2 ± 3.03 |
| | | | devices | | |
| 0.1 | 8 | Normal | Fresh | 16.7 ± 4.5 | 1.5 ± 0.1 |
| | | NH₄OH | samples | | |
| | | addition | | | |
| | | | Measured | 38.7 ± 37.9 | 2.0 ± 1.4 |
| | | | devices | | |
| 0.1 | 8 | Shaken with | Fresh | 15.0 ± 0.1 | 1.9 ± 0.01 |
| | | NH₄OH by | samples | | |
| | | Vortex | | | |
| | | | Measured | 19.0 ± 7.8 | 1.9 ± 0.1 |
| | | | devices | | |

### Conclusions

The present study indicates why the MPTMS layer deposited on the GaAs devices failed to protect. Apparently, pinholes are formed in the layer due to the increased temperature of the operated device. Solutions penetrating through these pinholes etch the GaAs surface of the devices and eventually cause them to malfunction. The new procedure disclosed herein provides a more uniform coating with better adhesion to the GaAs substrate (as a result, the effect of the temperature is less dramatic, allowing continuous electrical measurements in physiological solutions for more than 15 hours), and it thus makes it possible to operate the sensor in aqueous environments even at very low pHs for periods exceeding 24 hours (data not shown). Additional modifications of the procedure with better dispersion of NH₄OH acting as a condensation agent for polymerization result in a reduced thickness of the protecting layer, which ensures significantly higher sensitivity since the reduction in the signal is proportional to the thickness of the MPTMS layer. However, in these cases the protection is stable for 7 hours only.

### Study 2. MOCSER Devices Protected According to the Procedure Disclosed Herein are Highly Sensitive for Continuous Monitoring of Hemoglobin

### GaAs Device

The sensor used in this study is a GaAs-based MOCSER protected according to the new procedure described in the Experimental section, with sheep anti-human hemoglobin antibodies immobilized on its surface and serving as specific receptors for the hemoglobin molecules present in the analyte solution. Hence, the current between source and drain of the MOCSER device is controlled by the hemoglobin molecules interacting with the antibodies adsorbed on its surface (Tatikonda et al., 2013). The sensitivity of MOCSER device is achieved by applying a GaAs pseudomorphic high-electron mobility transistor configuration. The conducting channel of the device acts as a very thin layer of 2D electron gas and its conductivity is highly sensitive to changes in surface potential.

Surface passivation techniques for GaAs have been frequently reported but none of them have proven to be really compatible or stable enough for prolonged measurements in aqueous environments (Yi et al., 2007; Huang et al., 2005; Ohno and Shiraishi, 1990). Ultra-thin polymer coating chemically deposited by a sol-gel process with MPTMS was shown to significantly increase the long-term stability of GaAs surfaces (Hou et al., 1997) and, moreover, to be effective in protecting GaAs-based MOCSER devices operating in biological environments (Bavli et al., 2012). In the present study we thus adopted the basic concept; however, modified the procedure to create a more robust polymer coating capable of effective transferring the potential change on its surface to the GaAs substrate.

In particular, the substrate was etched with HF and NH₄OH in order to remove the oxide layer and to expose the arsenic-rich surface. MPTMS molecules were first adsorbed as a monolayer, by binding to the substrate through thiol ends and exposing silane groups. Next, NH₄OH was added to the solution inducing MPTMS polymerization and the formation of a dense polymer layer on the GaAs surface. The resulting polymer layer thickness was about 25-30 nm as estimated by ellipsometry. Such a polymer coating is stable for more than a week in deionized water and no increase in the oxide thickness on the GaAs surface is observed. Lastly, an APTMS layer was vapor deposited on top of the MPTMS layer for further binding of biological molecules.

### Surface Chemical Modification

For any bio-sensor, keeping nonspecific interactions of the analyte at minimum is a crucial factor in defining its sensitivity and selectivity (Frederix et al., 2004; Choi and Chae, 2010). For reducing false positive responses, two surface modification strategies were combined. First, oriented hemoglobin antibodies were anchored to the surface through protein G. As APTMS amine groups are positively charged, the negatively charged Protein G can be easily immobilized by electrostatic interaction on a GaAs/MPTMS/APTMS surface. It should further be noted that Protein G binds only the Fc terminal of the antibody and it thus provides a preferred orientation of the antibody epitopes (Fab terminals) away from the surface, exposing the antigen-binding site to the analyte (Lindman et al., 2006). Next, bare sites on the substrate, not coated with antibodies, were blocked by bovine serum albumin (BSA) so as to eliminate nonspecific interaction of the analyte with the substrate (Jung et al., 2007), thus achieving specific recognition and binding of the analyte to the immobilized antibody. The whole process of adsorbing Protein G, immobilizing the antibodies, surface blocking by BSA, and detecting hemoglobin present in the analyte solution can be followed by monitoring the change in current through the MOCSER devices.

### Measurement Procedure

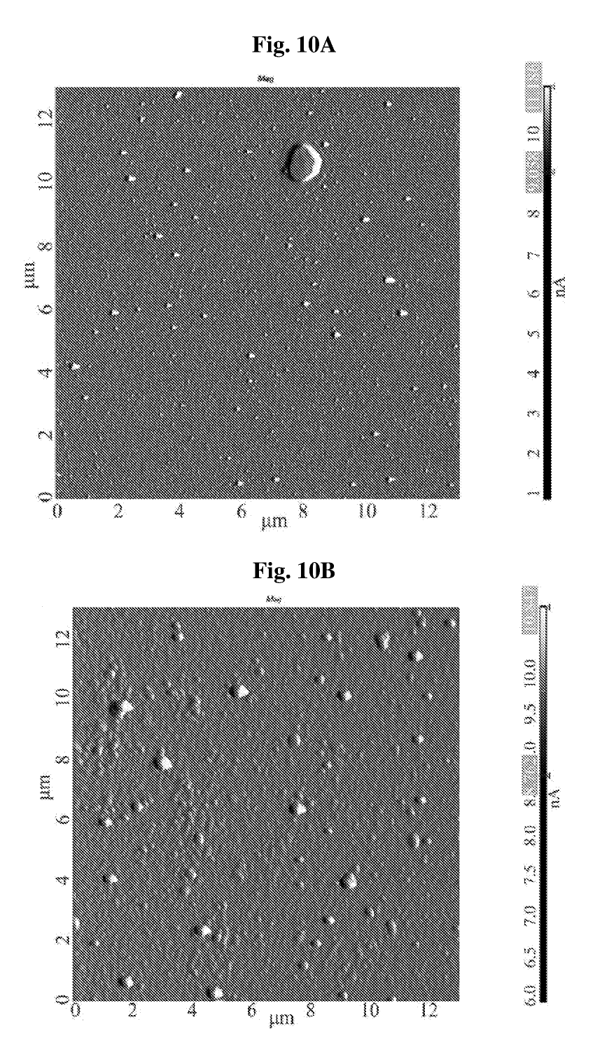

A PDMS-based microfluidic flow cell as described in the Experimental section was fixed on top of the MOCSER device in order to supply the analyte solutions and allow incubation in a controllable way (FIG. 11). Analytes were dissolved in either a phosphate buffer or a physiological fluid, and were injected sequentially into the flow cell. Phosphate buffer (50 mM; pH 7.4) used as the washing buffer was injected sequentially between analytes in order to wash the sensing area. The signal measured during this time was used as a baseline in the data analysis. A syringe pump (Hamilton, model: PHD) was used to ensure a controllable flow of small volumes of analytes.

Electrical measurements were performed on wire-bonded devices using Keithley 236 source-measure units and a Keithley 2700 switch control, controlled and monitored by a Labview application. An Ag/AgCl reference electrode was connected via a salt bridge to maintain a stable and constant potential over the surface of the MOCSER device. A constant voltage of 1.0 V was applied between the source and drain of the MOCSER device upon insertion into the microfluidic channel, and the change in source-drain current was monitored as a function of time while the analytes are introduced into the system (FIG. 11).

### Results

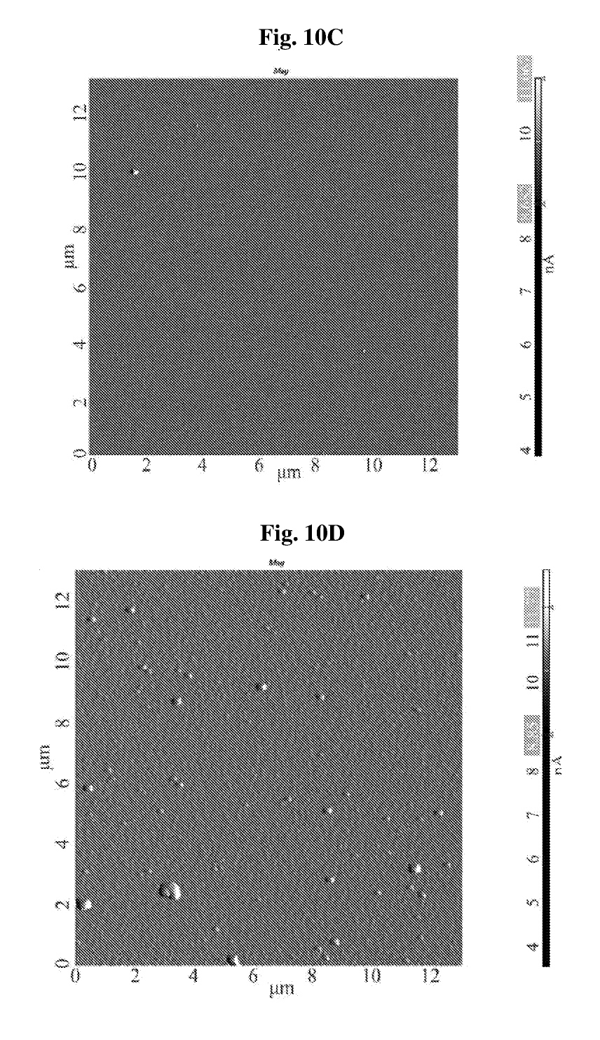

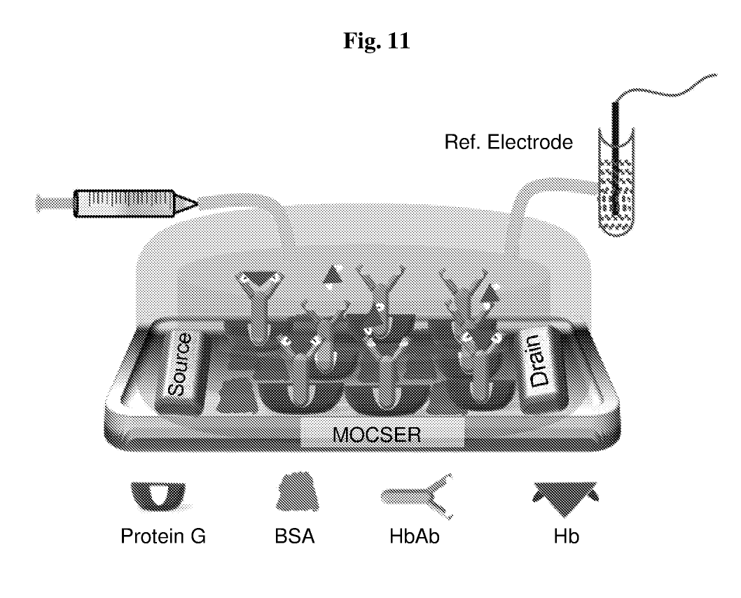

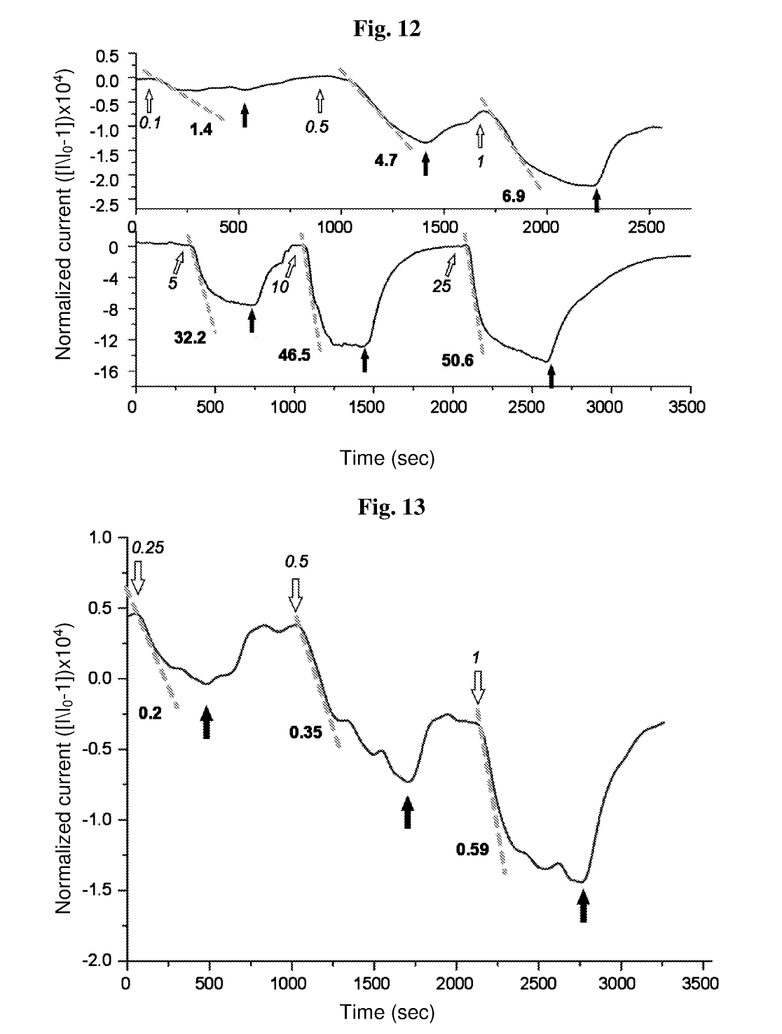

FIGS. 12 and 13 show the change in the source-drain current through the MOCSER device upon exposure to hemoglobin solutions with concentrations of 0.1, 0.5, 1, 5, 10 and 25 mg/ml dissolved in phosphate buffer (50 mM), or to hemoglobin solutions with concentrations of 0.25, 0.5 and 1 mg/ml dissolved in urine, respectively. As shown, the device response was immediate and stable upon exposure to the hemoglobin solution, wherein the current decreased when hemoglobin interacted with the antibody-modified surface and recovered after the hemoglobin was washed with the buffer or urine. The signal was correlated with the concentration of the analyte molecules, and a calibration plot was obtained by plotting the slope of the signal as a function of time vs. the hemoglobin concentration (FIG. 14). Since during the measurement all parameters beside hemoglobin concentration were kept constant, the slope of the signal upon changing the concentration was found to be proportional to the concentration of the analyte.

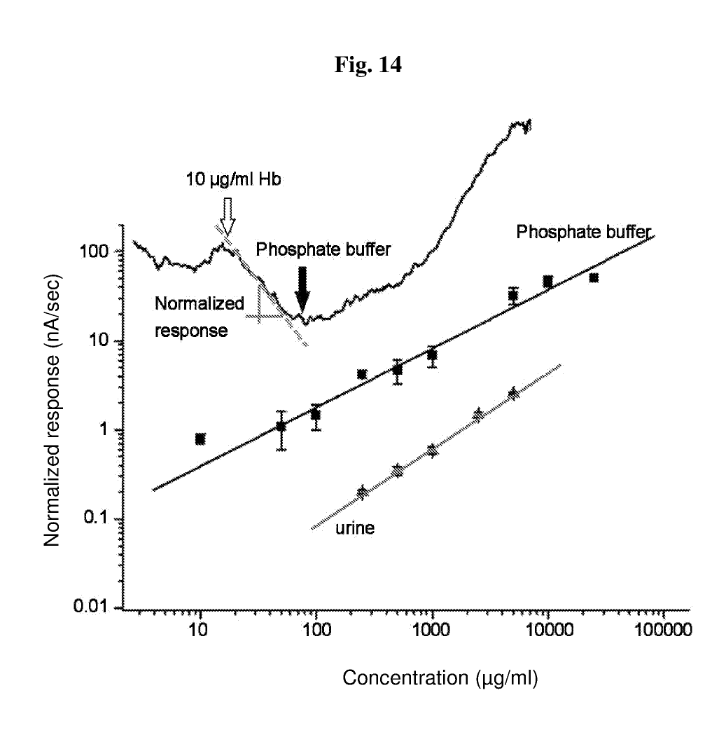

The ability of the sensor to detect hemoglobin in a very harsh environment was demonstrated by dissolving hemoglobin in physiological fluid collected during Endoscopic Retrograde Cholanigo Pancreatography (ERCP) from the duodenum, which is rich in bile juice (gastric intestine fluids). A known amount of hemoglobin was dissolved in the fluid after the latter was diluted with water 25-fold (the dilution was required to obtain the response; see hereinafter), and the results are shown in FIG. 15.

### Selectivity and Sensitivity

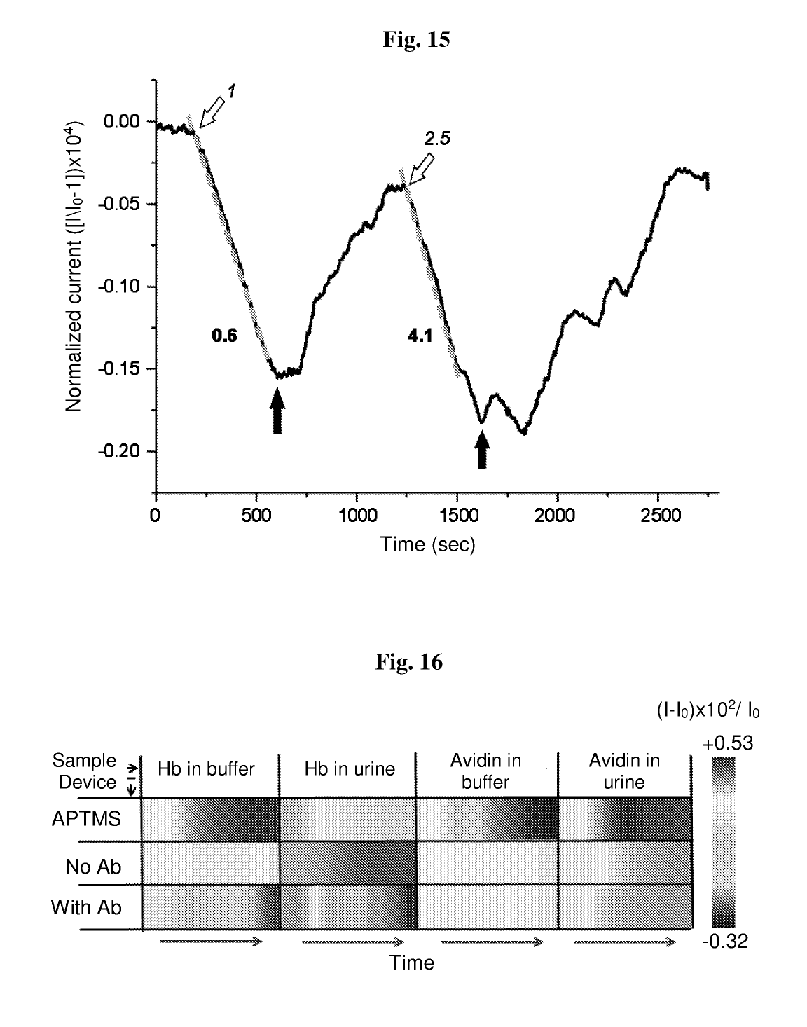

In order to investigate the selectivity of the MOCSER device, the source-drain currents as a function of time were measured for several devices with different surface modifications, operating as an array, upon exposure to different analytes, as shown in FIG. 16. An array unit to which the hemoglobin antibodies were not attached (the surface was functionalized with Protein G and BSA only) showed no response to hemoglobin. A device modified with Protein G-BSA-antibodies exhibited a strong response to hemoglobin both in the buffer and in urine, whereas the response to avidin (nonspecific to antibodies) was negligible. In contrast, a device coated with MPTMS-APTMS only was sensitive to avidin dissolved in the buffer with no significant response to hemoglobin analytes. Hence, by combining three units with different receptors exposed towards the analyte solution, extra selectivity was gained. The array can be easily expanded to many more units so as to overcome selectivity problems in various environments and for various analytes.

Monitoring the gradient instead of the net change in current ensures better reproducibility of the response and eliminates the contribution from baseline shifts. The sensitivity of the sensor towards hemoglobin was 10 μg/ml in phosphate buffer and 100 μg/ml in urine. The lower sensitivity to hemoglobin in urine, as compared to that in the phosphate buffer, results from the remarkably higher salt concentration in urine which screens the change in the electrical potential. The sensitivity to hemoglobin in ERCP was much lower than that in the buffer solution or urine, most probably due to denaturation of the hemoglobin antibodies (receptors) by the proteins/enzymes present in the bile juice. When the bile juice was diluted, the rate of denaturation was slow enough to allow binding of the hemoglobin to the receptors. This practice of dilution can be performed, in principle, also in vivo, as the volume of bile juice is typically 50 ml and by drinking several glasses of water it becomes significantly diluted.

### Sensing Mechanism

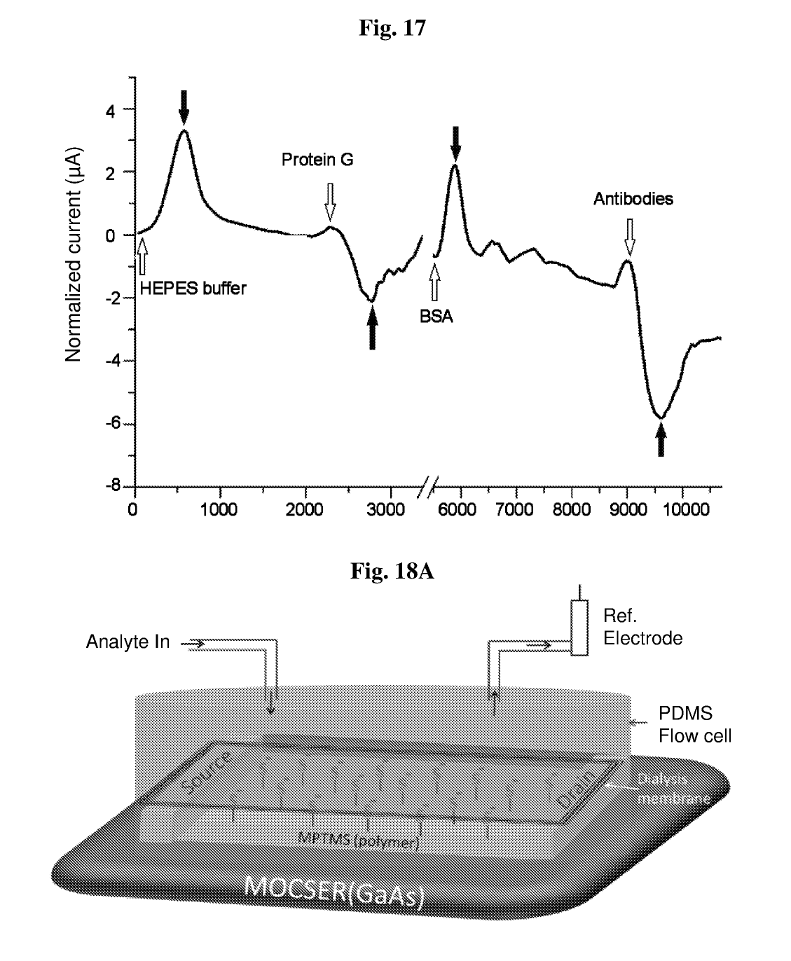

The sensing technology of the MOCSER is substantially different than those of the common ion-sensitive field effect transistor (ISFET) (Ghafar-Zadeh et al., 2010; Bergveld, 2003) and chemical field effect transistor (ChemFET) devices (Sibbald, 1983). In ISFET-based devices, a specific ion-selective permeable membrane replaces the metal gate area and allows the penetration of specific ions which define the electric potential of the gate. FIG. 17 demonstrates the change in the current in a MOCSER device upon exposure to Protein G, BSA and hemoglobin antibodies, and show that the net change in the current is negative when Protein G is introduced into the sensing area; positive upon introducing BSA; and negative when hemoglobin antibodies interact with the surface of the device. According to the theory of capacitive sensing in ISFETs (Ghafar-Zadeh et al., 2010), when a negative charge accumulates on the surface of the device, it attracts positive charges in the gated area which, in turn, populates the conduction channel with more electrons, leading to a rise in the current between the source and drain, and vice versa. In the present case, Protein G, BSA, hemoglobin antibodies, as well as hemoglobin are all negatively charged protein molecules at pH 7.4; however, exhibit different behavior in terms of change in current, demonstrating that the sensing mechanism of the MOCSER device is indeed different from that of ISFETs. ChemFET devices have a metal gate terminal coated with molecules interacting with a specific analyte, wherein the detection is performed by monitoring the change required in the gate potential so as to maintain a constant current upon exposure to the analyte. However, both technologies (ISFET and ChemFET) suffer from relatively low sensitivity.

In MOCSER devices, the current through the device for a given source-drain potential is determined by the resistivity which is directly controlled by the band bending in the semiconductor. In gate-less GaAs-based semiconductor devices, the band bending is determined by the charge associated with the surface states. The density of surface states in GaAs is about 10¹³ states/cm² eV and it has been shown that the number of surface states is reduced upon adsorption of molecules on the surface (Chang et al., 1999; Lee et al., 2008). Hence, the sensitivity of the MOCSER device stems from the change induced in the surface state charges.

### Study 3. MOCSER Devices Protected According to the Procedure Disclosed Herein are Capable of Sensing Ammonia in Ex-Vivo Gastrointestinal Fluids

### GaAs Device

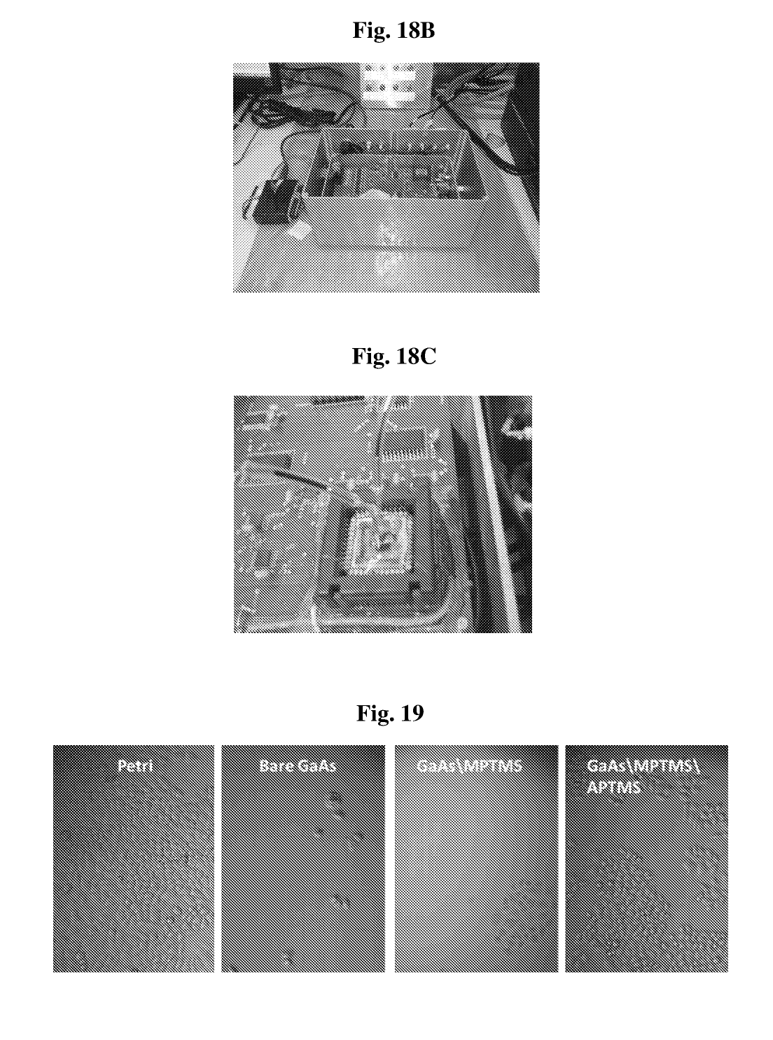

The sensor used in this study is a GaAs-based MOCSER protected according to the new procedure described in the Experimental section, wherein an APTMS layer is optionally deposited on top of the MPTMS polymer layer (as shown in Scheme 2) for further surface modification by using a sol-gel technique. The ultra-thin polymer of MPTMS passivates the surface from oxidation but also assigns a chemical functionality to the surface. Absorbing APTMS on the surface of the already polymerized MPTMS can be carried out electrostatically or nonspecifically, but this would result in leaching/washing away of APTMS from the surface during the measurements. In order to activate the hydroxyl groups of the MPTMS polymer, the samples with the MPTMS polymer were placed in the UVOC (UV ozone cleaner) for 20 seconds and immediately transferred into a 500 μl/ml of APTMS in ethanol. 75 μl/ml of NH₄OH was added to this solution to accelerate the condensation reaction and the solution was then incubated for 24 hours at room temperature. The polymer obtained had a thickness of about 4-5 nm measured by ellipsometry, and was quite stable even after washing for 24 hours in water. A semi-permeable cellulose based dialysis membrane with a 12 kDa cut-off molecular weight from Sigma (Cat: D9777) was used as a filter to eliminate the arrival of macromolecules on top the device's surface. Eventually, a PDMS-based microfluidic flow cell as described in the Experimental section was fixed on top of the MOCSER device in order to supply the analyte solutions and allow incubation in a controllable way (FIG. 18A).

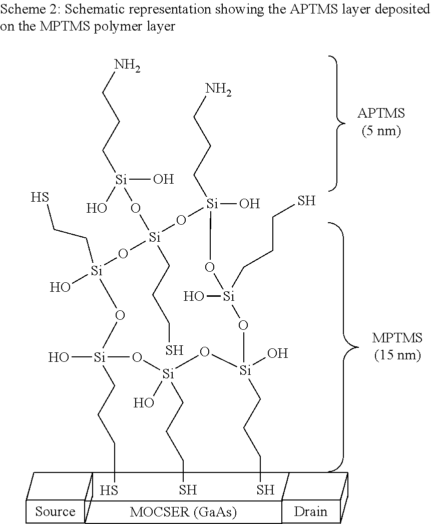 [SMILES: NCCC[Si](O)(O)O[Si]1(CCCS)O[Si](O)(CCS)O[Si](O)(CCCS)O[Si](O)(CCCS)O[Si](O)(CCCS)O[Si](O)(CCCS)O[Si](O)(CCCN)O1]

Ammonia was dissolved in solutions of different pH and in physiological fluids and was injected sequentially into the flow cell. A syringe pump (Harvard instruments) was used to ensure a controllable flow of precise volumes of analytes. Electrical measurements were performed on wire-bonded devices using a dual-channel source-measure unit (Keithley 2636A series) and a home built switching control box, controlled and monitored by a Labview application. An Ag/AgCl pseudo-reference electrode was connected via a salt bridge to maintain a stable and constant potential over the surface of the MOCSER device. A constant voltage of 1.0 V was applied between the source and drain of the MOCSER device, and the change in source-drain current was monitored as a function of time while the analytes are introduced into the system. Typically, an array of 16 devices was measured simultaneously.

A dual Faraday box with a circuitry board was developed in order to conduct low noise and stable measurements (FIG. 18B). The circuitry was built to measure simultaneously 40 different devices (FIG. 18C).

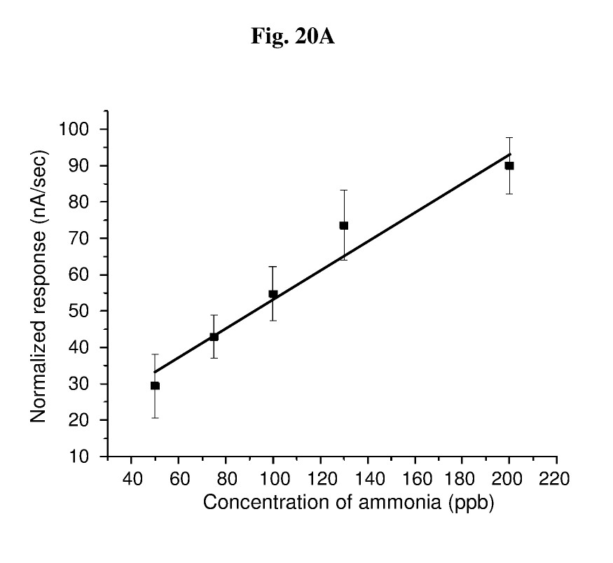

In order to prove the bio-compatibility of the sensor, human HeLa cells were cultured for 24 hours on petri dish, bare GaAs, GaAs coated with MPTMS, and GaAs coated with MPTMS/APTMS. While the cells did not grow on the bare GaAs, the coating of the GaAs with the polymers enables the growth of the cells (FIG. 19).

### Results

In order to verify the mechanism by which the polymer interacts with ammonia, the change in the current through the device upon exposure to solutions of various pH was monitored when the polymer is functionalized either with —SH (MPTMS) or —NH₂ (APTMS) groups. Experiments were performed in water in various pH for different ammonia concentration.

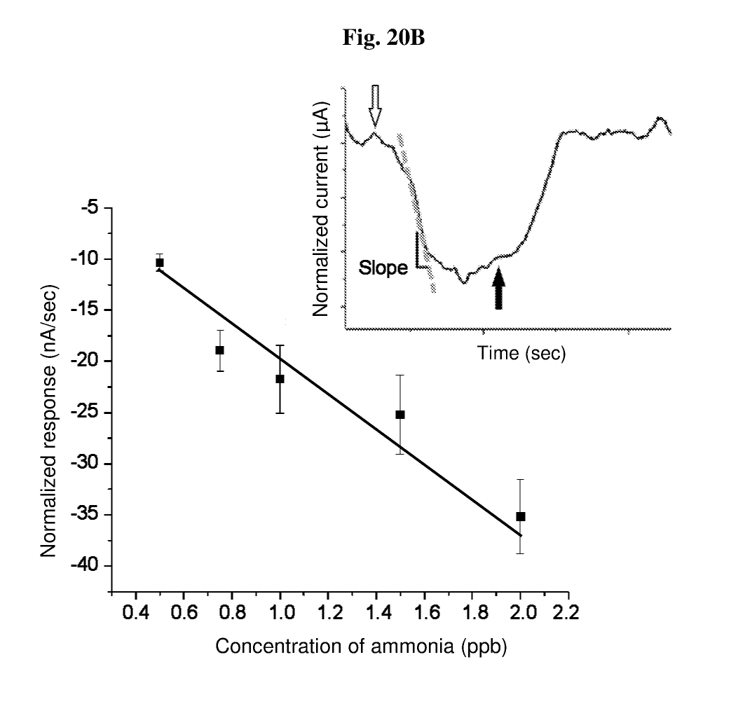

FIG. 20 shows a normalized response of the source-drain current through the MOCSER for different concentrations of ammonia in water at pH 1.2 (20A) and pH 5 (20B). Since during the measurement all parameters were kept constant and only the ammonia concentration was changed, the slope of the signal upon change in concentration was found to be proportional to the analyte concentration. The time response is related to the rate of injection and flow of the analyte and not to the actual response of the device.

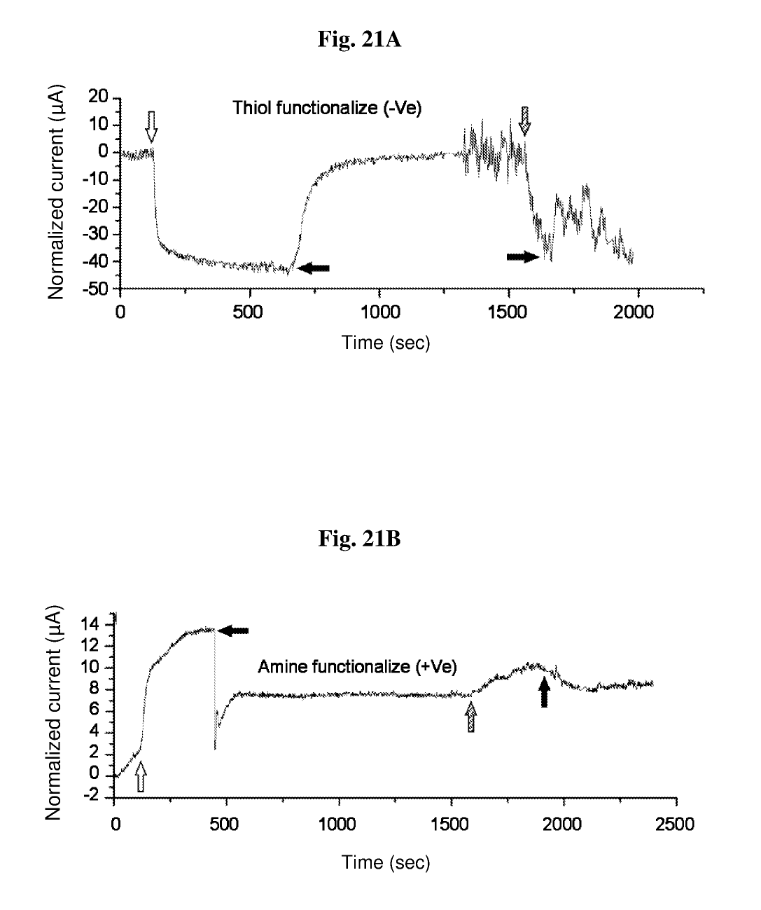

FIG. 21 shows the change in the response of the source-drain current through a MOCSER device having surface functionalized with MPTMS (21A) and APTMS deposited on top of the MPTMS (21B), when a known concentration of ammonia is added to water at pH 3, indicating the response to ammonia changes when the surface functionality is changed from MPTMS (—SH functionalized) to APTMS (—NH₂ functionalized).

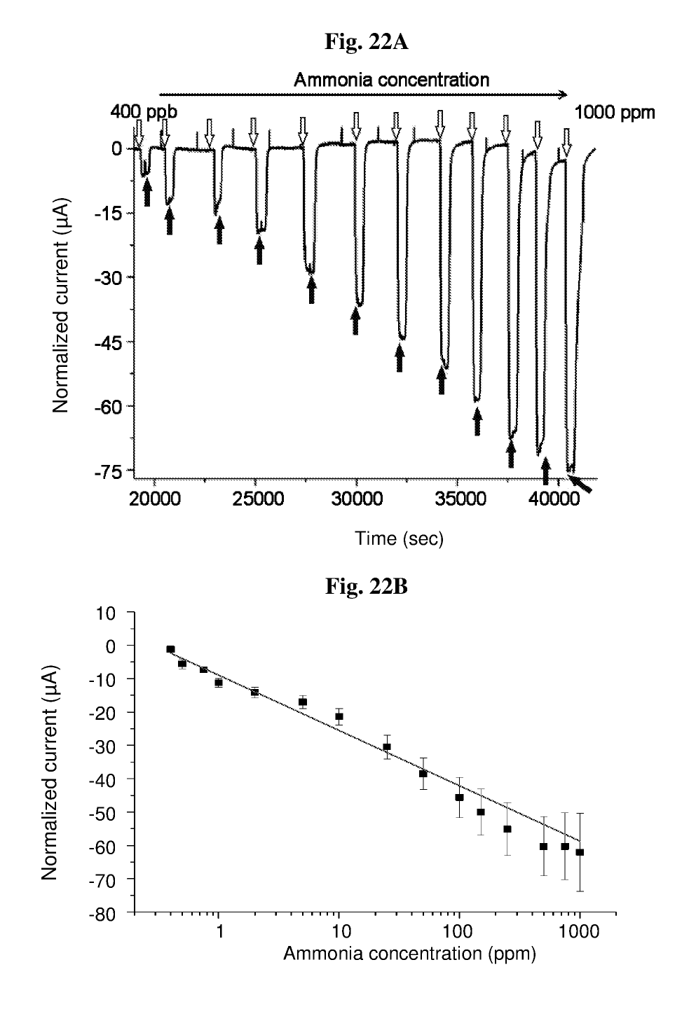

FIG. 22 shows the change in the current through the MOCSER coated with MPTMS upon exposure to different concentrations of ammonia. The dependence of the current on the concentration is clearly exponential and the minimal concentration that can be detected is below 400 ppb.

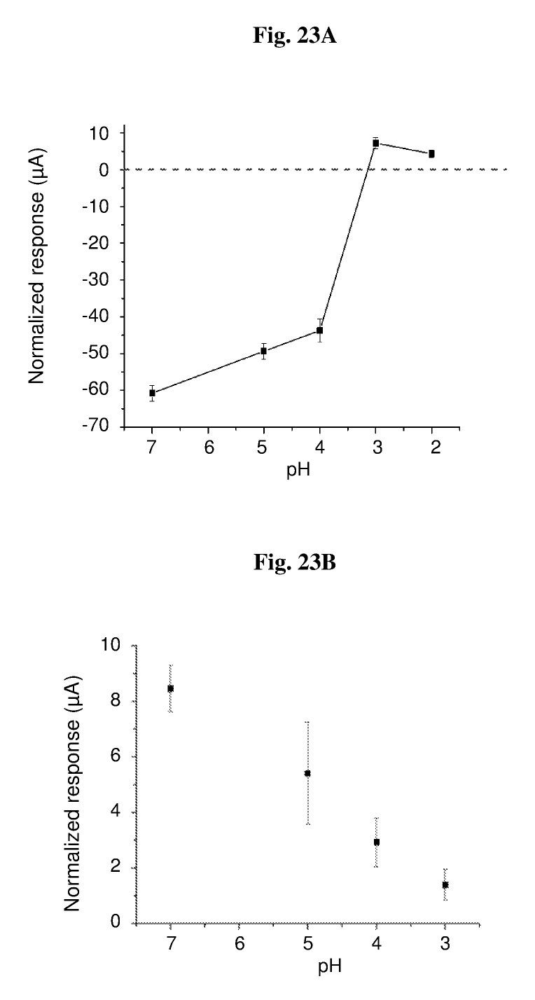

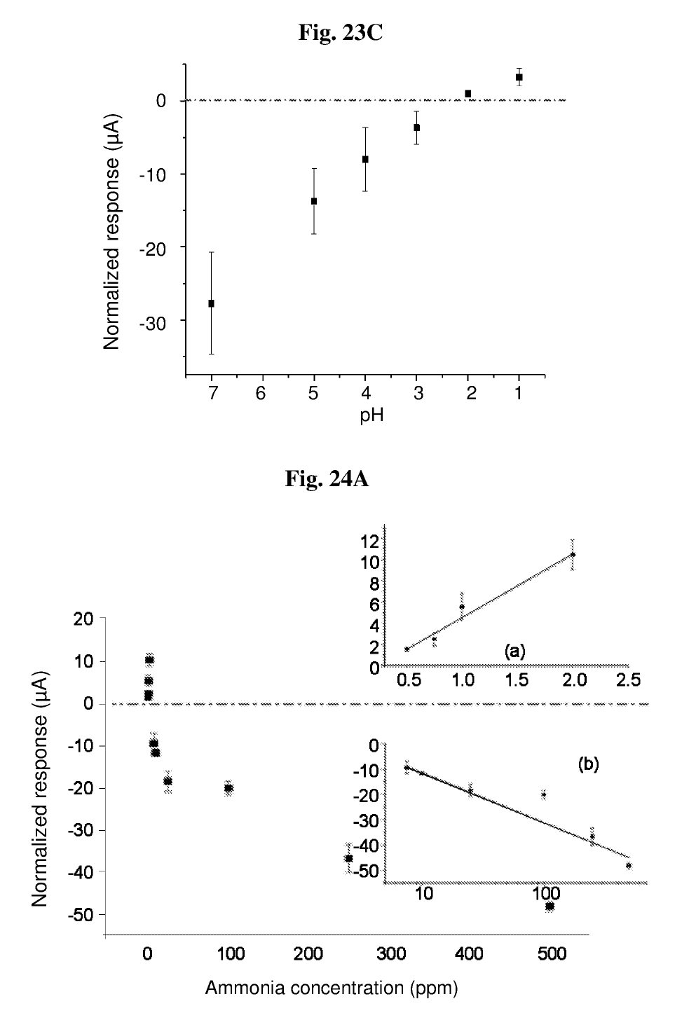

In order to demonstrate the mechanism by which the sensor response, devices coated either with APTMS or MPTMS were exposed to ammonia dissolved in water at different pH (FIG. 23). For devices coated with APTMS the response was positive, i.e., the current increases upon exposure to ammonia, while for those coated with MPTMS, the current decreases upon exposure to ammonia at low pH and becomes positive at pH 1. In both cases, the absolute change in the current decreases as pH becomes more acidic; however, for the MPTMS coated device, the response is very small at pH 2 and increases for pH 1. FIG. 24 shows the change in the current upon exposure of a device coated with APTMS or MPTMS to different concentrations of ammonia when the solution is at pH 4 (before addition of the ammonia). As shown in the insets, at low ammonia concentrations the response of the device is linear, while at higher concentrations it is logarithmic.

While the experiments described above aimed at establishing the sensing mechanism, the main challenge is to be able performing the measurements at realistic physiological conditions.

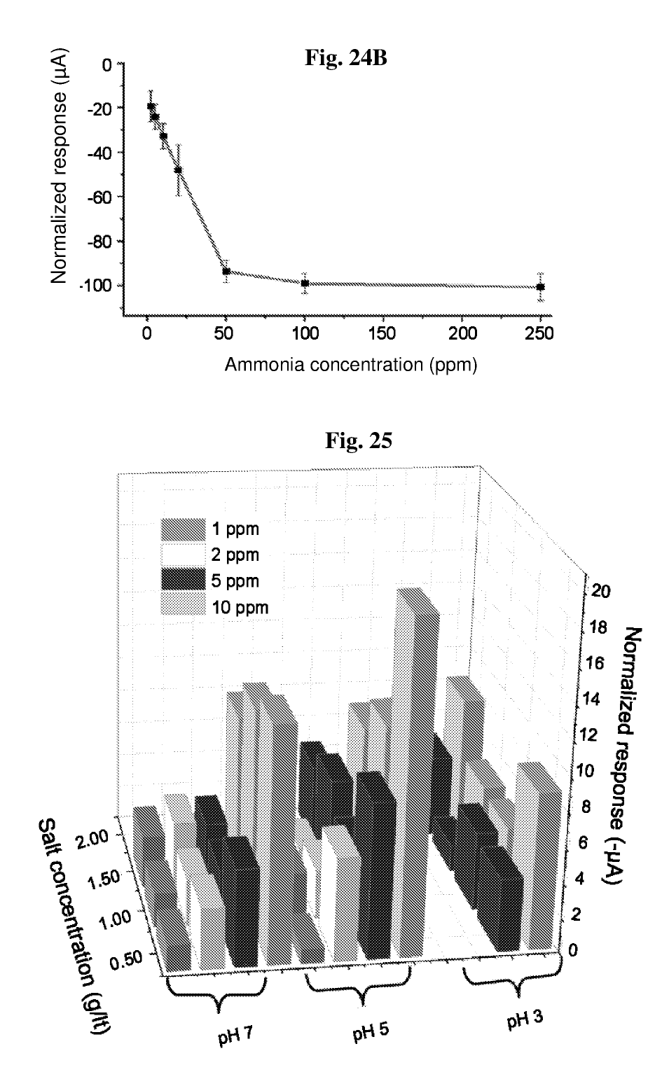

FIG. 25 summarizes the response of the sensor coated with MPTMS to ammonia in different gastric fluid simulation conditions. The parameters that were varied in the fluids are the salt concentration and the pH. As observed before, the device is more sensitive at higher pH; however, it functions well also at lower pH values and at different salt concentrations. The next test was performed in gastric fluids taken from patients to which known amount of ammonia was added. The sensor was washed with solutions of gastric simulation between measurements. In order to avoid the agglomeration of macromolecules on the surface of the sensor, a dialysis membrane was put on top of the MPTMS coated sensor.

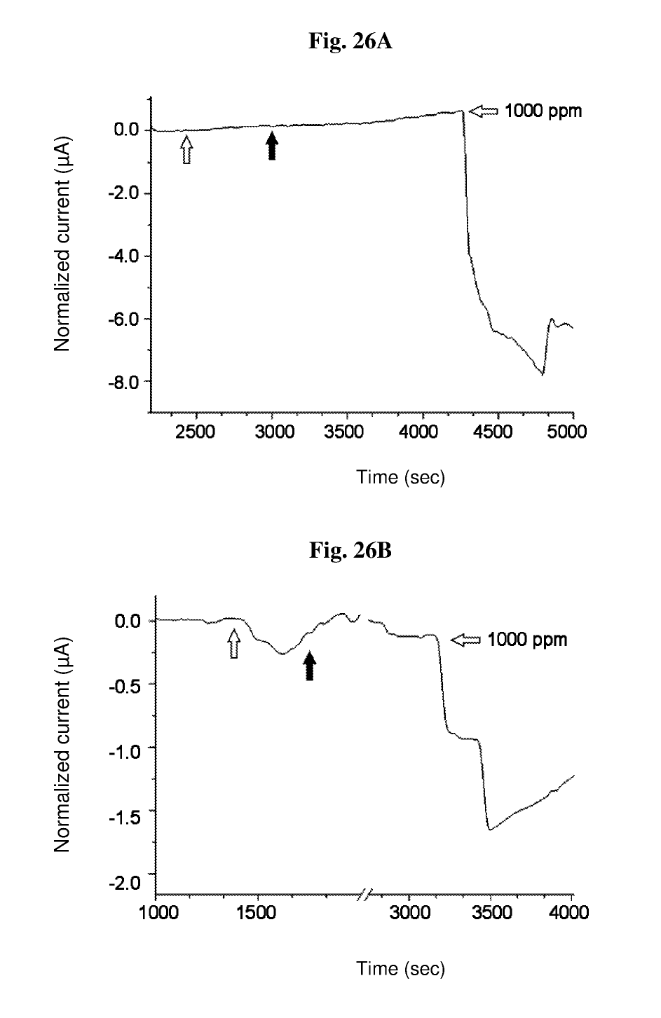

Finally, the sensor was applied for monitoring ammonia in gastro fluids obtained from both patients diagnosed with H. Pylori (+Ve) and healthy people (−Ve) not effected by H. pylori, using the esophagogastroduodenoscopy procedure (the measurement obtained with gastro fluid simulation was used as a baseline). In order to reduce evaporation of volatile materials like ammonia, the gastric fluids were immediately kept at −20° C. and defrosted only before the experiments. When −Ve was used as a baseline and +Ve as analyte, we didn't observe any response coming from the +Ve and only observed response when spike with a very high concentration of ammonia in +Ve (FIG. 26A). Gastric fluids contain large quantities of protease enzymes and HCl which contributes to denaturation of proteins, agglomeration of fatty acids and other carbohydrates resulting in a large quantity of macromolecules to precipitate and block the surface of the sensing are of the MOCSER. By filtering the gastric fluids externally by a 0.2 μm PTTE filter we started observing the responses from the +Ve and also from lower concentrations of ammonia added to the +Ve (FIG. 26B). To avoid external filtration of the gastric fluids from the patience we have used a dialysis membrane (10 kDa) on the top of the sensing area which would block large macromolecules and only allow small molecules like ammonia. Dialysis membrane being hydrophilic, the macromolecules can be easily washed away from the surface allowing the surface to recover and perform further measurements.

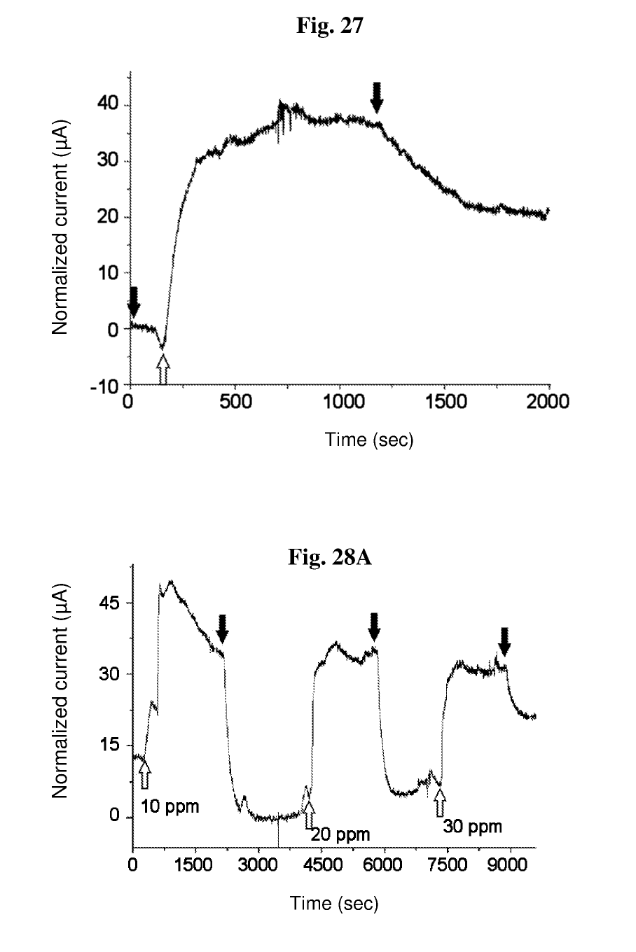

FIG. 27 shows a larger change for the positive sample vs. the negative one, wherein that change is by about one order of magnitude larger than the variation in the signal obtained among four negative samples.

### Discussion

The MOCSER is a semiconductor device sensitive to the electrical potential, φ, on its surface (Capua et al., 2009a). Namely, for constant physical parameters (device and system), the change in the channel's source-drain current, ΔI, upon exposure to the analyte, is determined by the surface/electrolyte interfacial potential. Utilizing the site binding theory (Yates et al., 1974) and based on the Nernst equation, the surface interfacial potential can be defined as (Bousse et al., 1983)

$$Δ I = αΔϕ = 2.3 \frac{α kT}{q} \{ \frac{β}{( β + 1 )} + γ [ {NH}_{3} ] \} ( pI - pH ) \qquad ( 1 )$$

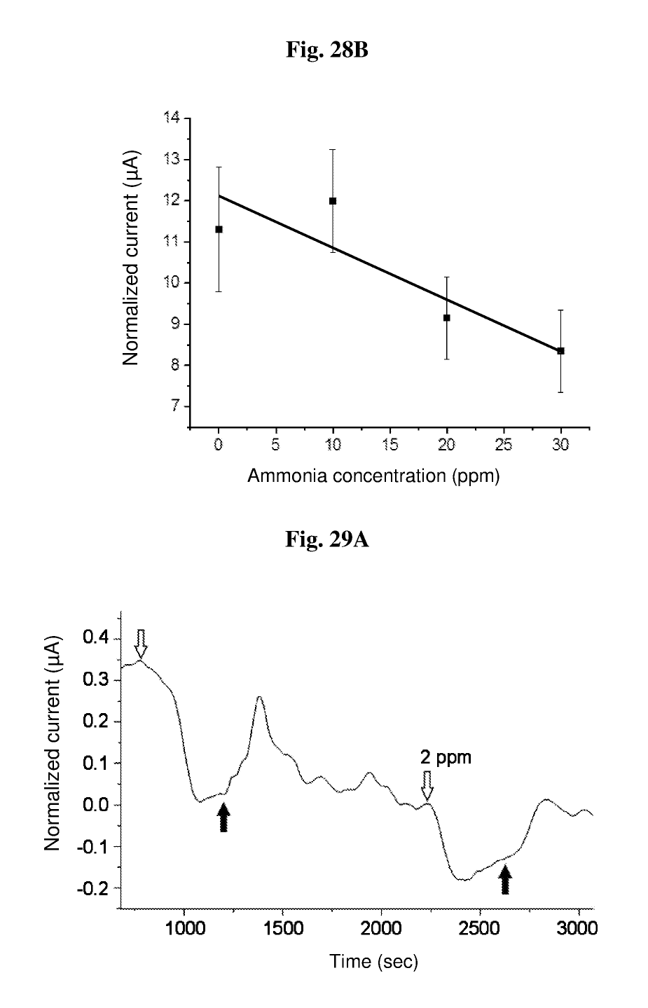

when α is the relation between the source-drain current and the surface potential (for relatively small range of φ, one can assume that a is constant); q is the surface charge; and β and γ are sensitivity parameter, where β it is the ratio of double layer formed by electrolyte to that of thermal potential and γ reflects the affinity to ammonia. The pH is defined for a given solution electrolyte/analyte and pI is the isoelectric point of the polymer coating the surface. When the pH of the analyte is higher or lower than the pI of the surface it protonates or deprotonates the surface, respectively. Isoelectric points were calculated from ChemAxon (Marvin sketch) simulations and are listed in Table 6. The pI of MPTMS polymer was found to be around 2.1 while for the APTMS polymer is was 9.7. FIG. 23 shows that when the APTMS-coated device is exposed to ammonia, the change in current is positive for all ranges of pH. When the coating is MPTMS, the change in the current is negative for pH higher than the pI, i.e., 2.1, and positive for pH lower than the pI. As a result of the dependence of the signal on pH we verified that while the lowest detectable concentration of ammonia in water (pH 7) is around 400 ppb, in pH 1 it is around 0.5 (FIG. 28).

TABLE 6
Isoelectric point (pI) of different ultra-thin
polymers deposited on the GaAs surface (calculated
by using simulation from ChemAxon service)

| The coating material | pI (single molecule) | pI (polymer) |
|---|---|---|
| MPTMS | 3.3 | 2.1 |
| APTMS | 9.8 | 10.2 |
| APTMS on MPTMS | — | 9.7 |

As clearly seen from equation 1, the sensitivity of the device is supposed to be larger for the case when the pH is very different than the pI. Indeed, for APTMS we verified that the sensitivity is much higher in higher pH than in the lower one. Another parameter that may affect the sensitivity is the salt (NaCl) concentrations that may screen the charged surface. As shown in FIG. 25, the pH of the analyte has a bigger effect on the response then the salt concentrations of the analyte.

Due to the proteases enzymes and HCl secreted into the stomach, the gastric fluids extracted from the patients have a large quantity of undissolved and precipitated carbohydrates, agglomerated fat, and denatured proteins. These macromolecules block the MOCSER sensing area when the gastro fluid is used as is, or the sensor surface is not protected. This results in no response of the sensor to ammonia (data not shown). Filtering out the macromolecules from the gastric fluid overcomes this problem. External filtration of gastric fluid collected from the patients is time consuming. This can be avoided by placing a filter on top of the sensing area. For this purpose, a 12 kDa semipermeable dialysis membrane was placed directly on the top of the MOCSER sensing area.

FIG. 28 presents the response from the unfiltered and undiluted gastric fluids. The sensitivity obtained with this configuration was of about 10 ppm, while without the membrane no response could be observed. The same response was observed for different samples of gastro fluids taken from 5 different patients. The error-bars in FIG. 28 reflect the variability in the response obtained with different patients. It is important to realize that gastric fluids taken from positive H. pylori patients are known to have at least 100 ppm of ammonia, hence the sensitivity obtained exceeds that required for detection of H. pylori. This statement is verified in FIG. 27 where results are shown for samples taken from a patient diagnosed as positive to H. pylori and another one diagnosed as negative. A clear different is the response of the device is observed between these two samples.

### Conclusions

The ability to apply semiconductor devices for sensing metabolites in biological environments opens up the possibility of taking advantages of the microelectronic-based technologies in real-time applications for sensing various molecules in the physiological environment, with no need to treat the samples before conducting the analysis. The devices could function for hours within the physiological solution without being damaged. In the present work it is demonstrated that ammonia can be sensed in gastro-fluids at pH 1.2 with sensitivity of 10 ppm. The blocking of the sensing area by undissolved and precipitated macromolecules was eliminated by placing a dialysis membrane. We demonstrated that the surface can be modified by two different molecules showing different responses and hence by constructing an array of MOCSERs with different surface functionalities it is possible to eliminate nonspecific interactions and false positive results. The combination of several different sensing elements allows the detection of ammonia with no need to pre-calibrate the individual devices.

The sensor developed combines sensitivity and selectivity with short measuring time and low production costs and therefore is an attractive venue for continuous sensing of ammonia.

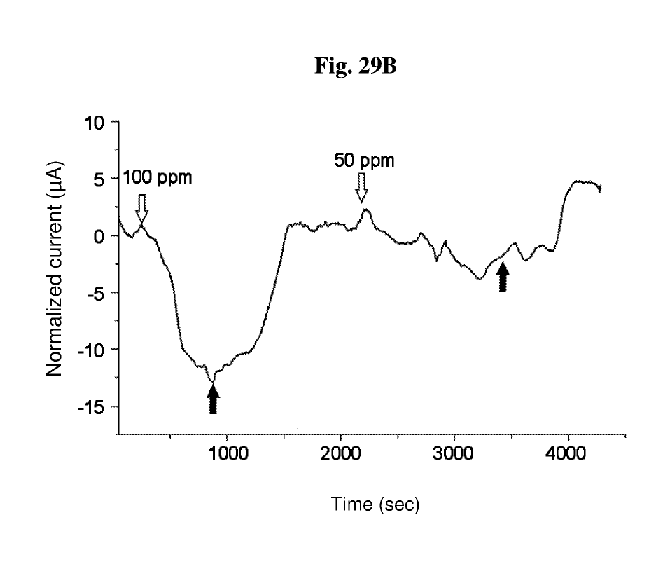

Monitoring the gradient instead of the net change in current ensures better reproducibility of the response and eliminates the contribution of the baseline shifts. The sensitivity of the sensor towards ammonia was 400 ppb in water, but only 10 ppm in gastric fluids (pH 1), most probably due to the screening of ammonia by other macromolecules present in gastric fluids. Due to the enzymes (proteases) and HCl secreted into the stomach, the gastric fluids extracted from the patients have a large quantity of undissolved and precipitated carbohydrates, agglomerated fats and denatured proteins. These macromolecules block the MOCSER sensing area when exposed to the analyte solution, resulting in a negligible response (data not shown); however, this problem was overcome by filtering out the macromolecules from the gastric fluids, as shown in FIG. 29A. The filtration could be avoided by placing a membrane on the MOCSER sensing area which is permeable only to ammonia, as demonstrated in FIG. 29B, showing the response for unfiltered and undiluted gastric fluids when a 10 kDa dialysis membrane was placed on the MOCSER.

### REFERENCES

Alegret, S., Alonso, J., Bartroli, J., Del Valle, M., Analytical Chemical Acta, 1990, 231, 53-58
Aqua, T., Naaman, R., Aharoni, A., Banin, U., Paltiel, Y., Applied Physics Letters, 2008, 92, 223112-3
Arregui, F. J. Springer, 2009
Arudra, P., Marshall, G. M., Liu, N., Dubowski, J. J., J. Phys. Chem. C, 2012, 116, 2891-2895
Autret-Leca, E., Jonville-Béra, A. P., Llau, M. E., Bavoux, F., Saudubray, J. M., Laugier, J., Devictor, D., Barbier, P., J. Clin. Epidemiol., 2001, 54, 857-862
Bavli, D., Tkachev, M., Piwonski, H., Capua, E., de Albuquerque, I., Bensimon, D., Haran, G., Naaman, R., Langmuir, 2012, 28, 1020-1028
Bekyarova, E., Davis, M., Burch, T., Itkis, M., Zhao, B., Sunshine, S., Haddon, R., Journal of Physical Chemistry B, 2004, 108, 19717-19720
Bergveld, P., Biosensors, 1986, 2, 15-33
Bergveld, P., Sensor. Actuat. B-Chem., 2003, 88(1), 1
Bessolov, V. N., Lebedev, M. V., Ivankov, A. F., Bauhofer, W., Zahn, D. R. T., Applied Surface Science, 1998, 133, 17-22
Bousse, L., Rooi J, N. F., Bergveld, P., IEEE Transactions on electronic devices, 1983, ED-30(10), 1263-1270
Capua, E., Natan, A., Kronik, L., Naaman, R., ACS Appl. Mater. Interfaces, 2009a, 1, 2679-2683
Capua, E., Cao, R., Sukenik, C. N., Naaman, R., Sensors and Actuators B: Chemical, 2009b, 140, 122-127
Chang, G. S., Hwang, W. C., Wang, Y. C., Yang, Z. P., Hwang, J. S., J. App. Phy., 1999, 86, 1765
Chittajallu, R., Neithercut, W., Macdonald, A., McColl, K., Gut, 1991, 32, 21-24
Choi, S., Chae, J., J. Micromech. Microeng., 2010, 20, 075015
Dahlin, A. B., Dielacher, B., Rajendran, P., Sugihara, K., Sannomiya, T., Zenobi-Wong, M., Vörös, J., Analytical and Bioanalytical Chemistry, 2012, 402, 1773-1784
Delagebeaudeuf, D., Delescluse, P., Etienne, P., Laviron, M., Chaplart, J., Linh, N. T., Electronics Letters, 1980, 16, 667-668
Ding, X., Dubowski, J. J., SPIE Proceedings, 2005, 5713, 545-551
Fraticelli, Y. M., Meyerhoff, M., Analytical Chemistry, 1981, 53(12), 1857-1861
Frederix, F., Bonroy, K., Reekmans, G., Laureyn, W., Campitelli, W., Abramov, M. A., Dehaen, M., Maes, G., J. Biochem. Biophys. Meth., 2004, 58, 67
Gartsman, K., Cahen, D., Kadyshevitch, A., Libman, J., Moav, T., Naaman, R., Shanzer, A., Umansky, V., Vilan, A., Chemical Physics Letters, 1998, 283, 301-306
Ghafar-Zadeh, E., Sawan, M., Chodavarapu, V. P., Hosseini-Nia, T., T. IEEE Trans. Biomed. Circuits Syst., 2010, 4, 232
Green, A. M., Spicer, W. E., Journal of Vacuum Science & Technology A, 1993, 11, 1061-1069
Hou, T., Greenlief, C. M., Keller, S. W., Nelen, L., Kauffman, J. F., Chem. Mater., 1997, 9, 3181-3186
Huang, X., Lindgren, E., Chelikowsky, J. R., Phys. Rev. B, 2005, 71, 165328
Humenyuk, I., Torbiéro, B., Assie-Souleille, S., Colin, R., Dollat, X., Franc, B., Martinez, A., Temple-Boyer, P., Microelectronics Journal, 2006, 37, 475-479
Jaffrezic-Renault, N., Wan, K., Senillou, A., Chovelon, J., Martelet, C., Analusis, 1999, 27(7), 578-586
Jung, Y., Lee, J. M., Jung, H., Chung, B. H., Anal. Chem., 2007, 79, 6534
Kirchner, C., George, M., Stein, B., Parak, W. J., Gaub, H. E., Seitz, M., Advanced Functional Materials, 2002, 12, 266-276
Konenkova, E. V., Vacuum, 2002, 67, 43-52
Lee, K., Lu, G., Facchetti, A., Janes, D. B., Marks, T. J., App. Phy. Lett., 2008, 92, 123509
Lindman, S., Xue, W. F, Szczepankiewicz, O., Bauer, M. C., Nilsson, H., Linse, S., Biophys. J., 2006, 90, 2911
Liu, H., Kameoka, J., Czaplewski, D., Craighead, H., Nano Letter, 2004, 4(4), 671-675
Lunt, S. R., Ryba, G. N., Santangelo, P. G., Lewis, N. S., Journal of Applied Physics, 1991, 70, 7449-7467
Makowski, M. S., Ivanisevic, A., Small, 2011, 7, 1863-1875
Megraud, F., Neman-Simha, E., Brugmann, D., Infection and Immunity, 1992, 60(5), 1858-1863
Meyerhoff, M., Robins, R., Analytical Chemistry, 1980, 52, 2388-2387
Meyerson, L. R., McMurtrey, K. D., Davis, V. E., Analytical Biochemistry, 1978, 86, 287-297
Mohabbati-Kalejahi, E., Azimirad, V., Bahrami, M., Ganbari, A., Talanta, 2012, 97, 1-8
Naaman, R., Phys. Chem. Chem. Phys., 2011, 13, 13153-13161
Nishiyama, N., Horie, K., Asakura, T., Journal of Colloid and Interface Science, 1989, 129, 113-119
Ohno, T., Shiraishi, K., Phys. Rev. B, 1990, 42, 11194
Packham, D. K., Perkovic, V., Savige, J., Broome, M. R., Semin Nephrol., 2005, 25, 146
Radomska, A., Koncki, R., Pyrzynska, K., Glab, S., Analytical Chimical Acta, 2004, 523, 193-200
Salimi, A., Hallaj, R., Khayatian, G.-R., Electroanalysis, 2005, 17(10), 873-879
Seker, F., Meeker, K., Kuech, T. F., Ellis, A. B., Chem. Rev., 2000, 100, 2505-2536
Senillou, A., Jaffrezic-Renault, N., Martelet, C., Cosnier, S., Talanta, 1999, 50, 219-226
Sibbald, A., IEE Proceedings I, 1983, 130(5), 233
Tatikonda, A. K., Tkachev, M., Naaman, R., Biosensors and Bioelectronics, 2013, 45, 201-205
Thévenot, D. R., Toth, K., Durst, R. A., Wilson, G. S., Biosensors and Bioelectronics, 2001, 16, 121-131
Vilar, M. R., El-Beghdadi, J., Debontridder, F., Artzi, R., Naaman, R., Ferraria, A. M., Do Rego, A. M. B., Surface and Interface Analysis, 2005, 37, 673-682
Vilar, M. R., El-Beghdadi, J., Debontridder, F., Naaman, R., Arbel, A., Ferraria, A. M., Do Rego, A. M. B., Materials Science and Engineering: C, 2006, 26, 253-259
Yates, D. E., Levine, S., Healy, T. W., Journal of the Chemical Society Faraday Transactions 1, 1974, 70, 1807-1818
Yi, X., Li, H. C., Ye, P. D., IEEE Trans. Electron Devices, 2007, 54, 1811

1. A semiconductor device comprising at least one conducting semiconductor layer, optionally at least one insulating or semi-insulating layer, and a protective organic molecular layer fabricated on top of an upper layer which is either one of said at least one conducting semiconductor layer or one of said at least one insulating or semi-insulating layer, protecting said upper layer from corrosion,
said protective organic molecular layer is configured such that when in contact with an aqueous solution, said semiconductor device is sensitive to pH changes in said solution both when fresh and following application of a constant electrical potential of 1 Volt through said at least one conducting semiconductor layer for a period of time of at least 10 hours.

2. A semiconductor device according to claim 1, comprising at least one conducting semiconductor layer, at least one insulating or semi-insulating layer, two conducting pads, and a protective organic molecular layer,
wherein said at least one conducting semiconductor layer is on top of one of said at least one insulating or semi-insulating layer, said two conducting pads are on both sides on top of an upper layer which is either one of said at least one conducting semiconductor layer or one of said at least one insulating or semi-insulating layer, making electrical contact with said at least one conducting semiconductor layer, and said protective organic molecular layer is fabricated on top of said upper layer.

3. A semiconductor device according to claim 2, comprising at least one insulating or semi-insulating layer, one conducting semiconductor layer, two conducting pads, and a protective organic molecular layer,
wherein said conducting semiconductor layer is on top of one of said insulating or semi-insulating layers, said two conducting pads are on both sides on top of an upper layer which is either said conducting semiconductor layer or one of said insulating or semi-insulating layers, making electrical contact with said conducting semiconductor layer, and said protective organic molecular layer is fabricated on top of said upper layer.

4. A semiconductor device according to claim 1, wherein each one of said conducting semiconductor layers independently is a semiconductor selected from a III-V and a II-VI material, or a mixture thereof, wherein III, V, II and VI denote the Periodic Table elements III=Al, Ga, In; V=As, P; II=Cd, Zn; VI=S, Se, Te.

5. A semiconductor device according to claim 4, wherein each one of said conducting semiconductor layers independently is doped GaAs, doped (Al,Ga)As, or doped (In,Ga)As.

6. A semiconductor device according to claim 1, wherein each one of said insulating or semi-insulating layers independently is a dielectric material selected from silicon oxide, silicon nitride or an undoped semiconductor selected from a III-V and a II-VI material, or a mixture thereof, wherein III, V, II and VI denote the Periodic Table elements III=Al, Ga, In; V=As, P; II=Cd, Zn; VI=S, Se, Te.

7. A semiconductor device according to claim 6, wherein said undoped semiconductor is undoped GaAs, undoped (Al,Ga)As, or undoped (In,Ga)As.

8. A semiconductor device according to claim 1, composed of a first insulating or semi-insulating layer of undoped GaAlAs which is on top of a first conducting semiconductor layer of doped GaAs, said first conducting semiconductor layer is on top of a second insulating or semi-insulating layer of undoped GaAlAs which is on top of a third insulating or semi-insulating layer of undoped InGaAs, said third insulating layer is on top of a fourth insulating or semi-insulating layer of GaAs, wherein on top of said first insulating or semi-insulating layer is a second conducting semiconductor layer of GaAs on top of which is an upper insulating or semi-insulating layer of GaAs, and said protective organic molecular layer is fabricated on top of said upper insulating or semi-insulating layer.

9. A semiconductor device according to claim 1, wherein said protective organic molecular layer comprises a primary layer and a secondary polymer layer,
wherein said primary layer comprises a silane moiety of the formula —S—R₁—Si(OH)₃, —S—R₁—Si(OH)₂O—, —S—R₁—Si(OH)(O—)₂, —S—R₁—Si(O—)₃, —NH—R₁—Si(OH)₃, —NH—R₁—Si(OH)₂O—, —NH—R₁—Si(OH)(O—)₂, —NH—R₁—Si(O—)₃, or a mixture thereof; said secondary polymer layer is obtained upon polymerization under basic conditions of an alkoxysilane of the formula HS—R₁—Si(OR₂)₃, H₂N—R₁—Si(OR₂)₃, R₁′—Si(OR₂)₃ or HSi(OR₂)₃, a tetraalkyl orthosilicate of the formula Si(OR₂)₄, a biotinylated form thereof, or a mixture of the aforesaid; R₁ each independently is a (C₁-C₇)alkylene, preferably (C₃-C₄)alkylene, more preferably propylene, optionally interrupted with one or more —NH— groups; R₁′ is a (C₁-C₇)alkyl, preferably (C₃-C₄)alkyl, more preferably propyl, optionally interrupted with one or more —NH— groups; R₂ each independently is a (C₁-C₄)alkyl, preferably methyl or ethyl; and said secondary polymer layer is covalently linked to the said primary layer via —Si—O— bonds.

10. The semiconductor device of claim 9, wherein said primary layer comprises (i) a silane moiety of the formula —S—R₁—Si(OH)₃, —S—R₁—Si(OH)₂O—, —S—R₁—Si(OH)(O—)₂, —S—R₁—Si(O—)₃, or a mixture thereof, wherein R₁ is —(CH₂)₃—; (ii) a silane moiety of the formula —NH—R₁—Si(OH)₃, —NH—R₁—Si(OH)₂O—, —NH—R₁—Si(OH)(O—)₂, —NH—R₁—Si(O—)₃, or a mixture thereof, wherein R₁ is —(CH₂)₃— or —(CH₂)₄—; or (iii) a silane moiety of the formula —NH—R₁—Si(OH)₃, —NH—R₁—Si(OH)₂O—, —NH—R₁—Si(OH)(O—)₂, —NH—R₁—Si(O—)₃, or a mixture thereof, wherein R₁ is —(CH₂)₂—NH—(CH₂)₃—.

11. A semiconductor device of claim 10, wherein said primary layer comprises a silane moiety of the formula —S—R₁—Si(OH)₃, —S—R₁—Si(OH)₂O—, —S—R₁—Si(OH)(O—)₂, —S—R₁—Si(O—)₃, or a mixture thereof, wherein R₁ is —(CH₂)₃—.

12. A semiconductor device according to claim 9, wherein said secondary polymer layer is obtained upon polymerization under basic conditions of 3-mercaptopropyltrimethoxysilane (MPTMS), 3-mercaptopropyltriethoxysilane, N¹-(3-(trimethoxysilyl)propyl)ethane-1,2-diamine, N¹-(3-(triethoxysilyl)propyl)ethane-1,2-diamine, 3-aminopropyltrimethoxysilane (APTMS), 3-aminopropyltriethoxysilane, 4-aminobutyltriethoxysilane, 4-aminobutyl trimethoxysilane, trimethoxypropylsilane, trimethoxyethylsilane, tetramethyl orthosilicate, a biotinylated form thereof, or a mixture of the aforesaid.

13. A semiconductor device according to claim 12, wherein said secondary polymer layer is obtained upon polymerization under basic conditions of MPTMS.

14. A semiconductor device according to claim 9, wherein said primary layer comprises a silane moiety of the formula —S—R₁—Si(OH)₃, —S—R₁—Si(OH)₂O—, —S—R₁—Si(OH)(O—)₂, —S—R₁—Si(O—)₃, or a mixture thereof, wherein R₁ is —(CH₂)₃—; and said secondary polymer layer is obtained upon polymerization under basic conditions of MPTMS.

15. A semiconductor device according to claim 14, wherein said protective organic molecular layer is formed by a process comprising the steps of:
(i) etching the upper surface of said upper layer;
(ii) immersing the etched surface of said upper layer in a solution of 0.1 vol. % MPTMS in ethanol, at a temperature of about 50° C. for about 8 hours, thereby forming a primary layer of MPTMS moieties deposited on top of said surface of said upper layer; and either:
a) immersing the surface of said upper layer on which a primary layer of MPTMS moieties is deposited in a solution of 0.3-0.4 vol. % MPTMS in ethanol, followed by the addition of a base such as NH₄OH to thereby initiate polymerization of said MPTMS and MPTMS moieties, at a temperature of about 50° C. for about 16 hours, thereby forming a secondary layer of polymerized MPTMS linked to the said primary layer via —Si—O— bonds; or
b) immersing the surface of said upper layer on which a primary layer of MPTMS moieties is deposited in a solution of 0.3-0.4 vol. % MPTMS and a base such as NH₄OH in ethanol, wherein said base initiates polymerization of said MPTMS and MPTMS moieties, at a temperature of about 50° C. for about 16 hours, thereby obtaining a secondary layer of polymerized MPTMS linked to the said primary layer via —Si—O— bonds.

16. A semiconductor device according to claim 15, wherein said protective organic molecular layer has (i) a thickness of 22.3±6.7 nm and a surface having an RMS roughness of 1.5±0.1 nm when fresh and RMS roughness of 2.0±1.4 following application of a constant electrical potential of 1 Volt through said at least one conducting semiconductor layer for a period of time of at least 10 hours; or (ii) a thickness of about 15.9±1.2 nm and a surface having an RMS roughness of 1.9±0.01 nm when fresh and RMS roughness of 1.9±0.1 nm following application of a constant electrical potential of 1 Volt through said at least one conducting semiconductor layer for a period of time of at least 10 hours.

17. A semiconductor device according to claim 9, wherein said protective organic molecular layer further comprises a tertiary layer deposited on top of said secondary polymer layer, wherein said tertiary layer comprises an alkoxysilane of the formula HS—R₁—Si(OR₂)₃, H₂N—R₁—Si(OR₂)₃, R₁′—Si(OR₂)₃ or HSi(OR₂)₃, a tetraalkyl orthosilicate of the formula Si(OR₂)₄, or a mixture of the aforesaid; R₁ each independently is a (C₁-C₇)alkylene, preferably (C₃-C₄)alkylene, more preferably propylene, optionally interrupted with one or more —NH— groups; R₁′ is a (C₁-C₇)alkyl, preferably (C₃-C₄)alkyl, more preferably propylene, optionally interrupted with one or more —NH— groups; R₂ each independently is a (C₁-C₄)alkyl, preferably methyl or ethyl; and said tertiary layer is covalently linked to the said secondary polymer layer.

18. A semiconductor device according to claim 17, wherein said alkoxysilane independently is MPTMS, 3-mercaptopropyltriethoxysilane, N¹-(3-(trimethoxysilyl)propyl)ethane-1,2-diamine, N¹-(3-(triethoxysilyl)propyl)ethane-1,2-diamine, APTMS, 3-aminopropyltriethoxysilane, 4-aminobutyltriethoxysilane, 4-aminobutyltrimethoxysilane, trimethoxypropylsilane, or trimethoxyethylsilane; and said tetraalkyl orthosilicate is tetramethyl orthosilicate.

19. A semiconductor device according to claim 18, wherein said primary layer comprises a silane moiety of the formula —S—R₁—Si(OH)₃, —S—R₁—Si(OH)₂O—, —S—R₁—Si(OH)(O—)₂, —S—R₁—Si(O—)₃, or a mixture thereof, wherein R₁ is —(CH₂)₃—; said secondary polymer layer is obtained upon polymerization under basic conditions of MPTMS; and a tertiary layer comprising APTMS is deposited on top of said secondary polymer layer.

20. A semiconductor device according to claim 2, for the detection of a target molecule in a solution, said device further comprising a layer of multifunctional organic molecules capable of binding said target molecule via a functional group thereof,
wherein said layer of multifunctional organic molecules is linked either directly or indirectly to said protective organic molecular layer, and exposure of said multifunctional organic molecules to a solution containing said target molecule causes a current change through the semiconductor device when a constant electric potential is applied between the two conducting pads.

21. A semiconductor device according to claim 2, for the detection of an active site-containing protein or a ligand thereof in a solution, said device further comprising said ligand or active site-containing protein,
wherein said ligand or active site-containing protein is linked either directly or indirectly to said protective organic molecular layer, and exposure of said ligand or active site-containing protein, to a solution containing said active site-containing protein or ligand, respectively, causes a current change through the semiconductor device when a constant electric potential is applied between the two conducting pads.

22. A semiconductor device according to claim 21, for the detection of said active site-containing protein, wherein said device comprises said ligand linked either directly or indirectly to said protective organic molecular layer, and exposure of said ligand to a solution containing said active site-containing protein causes a current change through the semiconductor device when a constant electric potential is applied between the two conducting pads.

23. A semiconductor device according to claim 21, for the detection of said ligand, wherein said device comprises said active site-containing protein linked either directly or indirectly to said protective organic molecular layer, and exposure of said active site-containing protein to a solution containing said ligand causes a current change through the semiconductor device when a constant electric potential is applied between the two conducting pads.

24. A semiconductor device according to claim 21, wherein (i) said active site-containing protein is an antibody, and said ligand is an antigen, or vice versa; (ii) said active site-containing protein is an enzyme, and said ligand is a substrate or inhibitor, or vice versa; (iii) said active site-containing protein is a receptor, and said ligand is a protein or organic molecule, or vice versa; or (iv) said active site-containing protein is a lectin, and said ligand is a sugar.

25. A semiconductor device according to claim 20, wherein said solution is an aqueous solution, a physiological solution, a bodily fluid such as amniotic fluid, aqueous humour, vitreous humour, bile, blood serum, breast milk, cerebrospinal fluid, cerumen (earwax), endolymph, perilymph, female ejaculate, gastric juice, mucus, peritoneal fluid, saliva, sebum (skin oil), semen, sweat, tears, vaginal secretion, vomit and urine, or a bodily fluid-based solution.

26. A method for the detection of a target molecule in a solution, said method comprising:
(i) exposing a semiconductor device according to claim 2 to said solution; and
(ii) monitoring the presence of said target molecules in said solution according to the changes in the current measured in said semiconductor device when a constant electric potential is applied between the two conducting pads.

27. The method of claim 26, wherein exposure of the functional groups of the alkoxysilane or tetraalkyl orthosilicate forming said secondary polymer layer or said tertiary layer deposited on top of said secondary polymer layer, if present, to a solution containing said target molecule causes a current change through the semiconductor device when a constant electric potential is applied between the two conducting pads.

28. The method of claim 27, wherein said target molecule is ammonia; said secondary polymer layer is obtained upon polymerization under basic conditions of an alkoxysilane of the formula HS—R₁—Si(OR₂)₃ or said tertiary layer, if present, comprises an alkoxysilane of the formula HS—R₁—Si(OR₂)₃; and exposure of the mercapto groups of said alkoxysilane to a solution containing ammonia causes a current change through the semiconductor device when a constant electric potential is applied between the two conducting pads.

29. A method for the detection of a target molecule in a solution, said method comprising:
(i) exposing a semiconductor device according to claim 20 to said solution; and
(ii) monitoring the presence of said target molecules in said solution according to the changes in the current measured in said semiconductor device when a constant electric potential is applied between the two conducting pads.

30. The method of claim 29, wherein exposure of said multifunctional organic molecules to a solution containing said target molecule causes a current change through the semiconductor device when a constant electric potential is applied between the two conducting pads.

31. The method of claim 26, for quantification of said target molecule in said solution, wherein the current change is proportional to the concentration of said target molecule in said solution.

32. A method for detection of an active site-containing protein or a ligand thereof in a solution, said method comprising:
(i) exposing a semiconductor device according to claim 21 to said solution; and
(ii) monitoring the presence of said active site-containing protein or ligand in said solution according to the changes in the current measured in said semiconductor device when a constant electric potential is applied between the two conducting pads.

33. The method of claim 32, wherein said active site-containing protein is hemoglobin, said ligand is hemoglobin antibody, and said hemoglobin antibody is linked either directly or indirectly to said protective organic molecular layer.

34. The method of claim 32 or 33, for quantification of said active site-containing protein or ligand thereof in said solution, wherein the current change is proportional to the concentration of said active site-containing protein or ligand thereof in said solution.

35. The method of claim 32, for studying receptor-ligand pair interactions, in particular, monitoring the interaction of a receptor in a solution with a ligand linked either directly or indirectly to said protective organic molecular layer, or vice versa.
